(19) United States
(12) Patent Application Publication
(10) Pub. No.: US 2024/0390417 A1
(43) Pub. Date: Nov. 28, 2024

(54) MNO NANOMATERIAL BASED INHIBITORS OF INFLAMMATION AND SARS-COV-2 VIRAL REPLICATION

(71) Applicants: Duke University, Durham, NC (US); Columbia University, New York, NY (US); NewSouth Innovations Pty Limited, Sydney (AU)

(72) Inventors: Smita NAIR, Durham, NC (US); Ibtehaj NAQVI, Durham, NC (US); Yiling ZHONG, New York, NY (US); Kam LEONG, New York, NY (US); Bruce SULLENGER, Durham, NC (US)

(21) Appl. No.: 18/697,398

(22) PCT Filed: Sep. 30, 2022
(86) PCT No.: PCT/US2022/077423

§ 371 Date: Mar. 29, 2024

Related U.S. Application Data
(60) Provisional application No. 62/250,311, filed on Nov. 3, 2015.
(60) Provisional application No. 63/305,340, filed on Feb. 1, 2022.
(60) Provisional application No. 63/343,156, filed on May 18, 2022.

(51) Int. Cl. A61K 33/32; A61K 47/69; A61P 37/06

(52) U.S. Cl. CPC A61K 33/32; A61K 47/6923; A61K 47/6929; A61P 37/06

(57) ABSTRACT

The present invention provides methods for using an anionic manganese oxide (MnO) nanoparticle-based nucleic acid scavenger to (1) inhibit viral infection of cells, (2) reduce or inhibit a viral infection of a subject, and (3) reduce viral infection induced inflammation in a subject.

### CROSS-REFERENCE TO RELATED APPLICATIONS

This application claims priority to U.S. Provisional Application No. 63/250,311 filed on Sep. 30, 2021, U.S. Provisional Application No. 63/305,340, filed on Feb. 1, 2022, and U.S. Provisional Application No. 63/343,156 filed on May 18, 2022, the contents of which are incorporated by reference in their entireties.

### STATEMENT REGARDING FEDERALLY SPONSORED RESEARCH

This invention was made with government support under grant number W81XWH1910463 awarded by the Department of Defense and under grant number RO1AR073935 awarded by the National Institutes of Health. The government has certain rights in this invention.

### BACKGROUND

Damage-associated molecular patterns (DAMPs) and pathogen-associated molecular patterns (PAMPs) are molecules that are released in response to cellular stress or tissue injury and that function as endogenous danger signals. DAMPs and PAMPs are detected by pattern recognition receptors, such as Toll-like receptors (TLRs), which initiate potent inflammatory responses by activating the innate immune system. Inflammation is an important part of the body's healthy response to injuries, diseases, and infections. However, overactivation of TLRs can ultimately lead to disruption of immune homeostasis and thus increase the risk for inflammatory and autoimmune diseases. Thus, methods that control TLR-mediated inflammation would aid in the treatment of many diseases.

One such disease is COVID-19, an infection caused by the virus severe acute respiratory syndrome coronavirus 2 (SARS-CoV-2). The symptoms of COVID-19 range from mild symptoms of upper respiratory infection to acute respiratory distress syndrome (ARDS) and respiratory failure requiring mechanical ventilation or extracorporeal membrane oxygenation. Patients at the severe end of the disease spectrum, such as those in the intensive care unit (ICU), can quickly deteriorate and are at high risk of mortality. Underpinning these poor outcomes is a hyperinflammatory response which contributes to the development and progression of lung injury and ARDS.

Vaccines and anti-SARS-CoV-2 monoclonal antibody-based therapies have been shown to be effective against SARS-CoV-2. But, unfortunately, all the available vaccines and therapies target the same SARS-CoV-2 spike protein, which is the region of the virus that is most prone to mutation. Because there is a population of people that is unable or unwilling to get vaccinated, this population will serve as a breeding ground for new variants of the virus, and we will need to continually develop and distribute new booster vaccines and therapies to keep these variants in check. Thus, a more universal approach to limiting the transmission and severity of infection of SARS-CoV-2 is needed.

### SUMMARY

In a first aspect, the present invention provides methods of reducing or inhibiting viral infection of cells. The methods comprise contacting the cells with a MnO nanoparticle described herein.

In a second aspect, the present invention provides methods of reducing or inhibiting a viral infection of a subject. The methods comprise administering a MnO nanoparticle described herein to the subject.

In a third aspect, the present invention provides methods of reducing inflammation in a subject. The methods comprise administering a MnO nanoparticle described herein to the subject.

### BRIEF DESCRIPTION OF THE DRAWINGS

FIG. 1 is a plot showing the measured hydrodynamic size of the anionic manganese oxide (MnO) nanoparticles generated by the inventors.

FIG. 2 is a graph comparing the cell viability of RAW264.7 cells treated with the MnO nanoparticles and PAMAM-G3 at varying concentrations.

FIG. 3A is a graph comparing the DNA binding affinity of the MnO nanoparticles and PAMAM-G3 at different material:DNA mass ratios in TE buffer.

FIG. 3B is a graph comparing inhibition of agonist-induced Toll-like receptor (TLR) activation by the MnO nanoparticles for HEK-Blue hTLR3 cells treated with fetal bovine serum (FBS) and untreated samples.

FIG. 3C is a graph comparing inhibition of agonist-induced TLR activation by the MnO nanoparticles for HEK-Blue hTLR8 cells treated with FBS and untreated samples.

FIG. 3D is a graph comparing inhibition of agonist-induced TLR activation by the MnO nanoparticles for HEK-Blue hTLR9 cells treated with FBS and untreated samples.

FIG. 4A is a graph comparing the hydrodynamic size of MnO nanoparticles, MnO@Curcumin nanoparticles, and MnO@Curcumin@IR780 nanoparticles.

FIG. 4B is a graph comparing the zeta potential of MnO nanoparticles, MnO@Curcumin nanoparticles, and MnO@Curcumin@IR780 nanoparticles.

FIG. 4C is a graph comparing the UV-visible light spectra of MnO nanoparticles, MnO@Curcumin nanoparticles, MnO@Curcumin@IR780 nanoparticles, IR780 iodide, and curcumin.

FIG. 4D is a graph comparing the photoluminescence (PL) spectra of MnO nanoparticles, MnO@Curcumin nanoparticles, MnO@Curcumin@IR780 nanoparticles, IR780 iodide, and curcumin.

FIG. 5A is a graph comparing the cell viability of RAW264.7 cells treated with MnO nanoparticles, MnO@Curcumin nanoparticles, and curcumin for 24 hours.

FIG. 5B is a graph comparing the cell viability of 4T1 cells treated with MnO nanoparticles, MnO@Curcumin nanoparticles, and curcumin for 24 hours.

FIG. 5C is a graph comparing DNA binding affinity of MnO nanoparticles, MnO@Curcumin nanoparticles, curcumin, and tannic acid at different nanoparticle:DNA mass ratios in a TE buffer without FBS.

FIG. 5D is a graph comparing the DNA binding affinity of MnO nanoparticles, MnO@Curcumin nanoparticles, curcumin, and tannic acid at different nanoparticle:DNA mass ratios in a TE buffer with 10% FBS.

FIG. 5E shows confocal laser scanning microscopy (CLSM) images of internalization of MnO nanoparticles by RAW264.7 cells and colocalization of Cy3-labeled MnO nanoparticles and Cy5-labeled cytosine-phosphate-guanosine oligodeoxynucleotide (CpG) in lysosomes (scale bar: 10 m).

FIG. 6A is a graph comparing the activation of HEK-Blue cells by MnO nanoparticles, MnO@Curcumin nanoparticles, and curcumin at different mass ratios with respect to CpG, without FBS (i.e., untreated (UT)) for 24 hours.

FIG. 6B is a graph comparing the activation of hTLR9 cells by MnO nanoparticles, MnO@Curcumin nanoparticles, and curcumin at different mass ratios with respect to poly (I:C), without FBS (i.e., untreated (UT)) for 24 hours.

FIG. 6C is a graph comparing the activation of hTLR3 cells by MnO nanoparticles, MnO@Curcumin nanoparticles, and curcumin at different mass ratios with respect to lipopolysaccharide (LPS), without FBS (i.e., untreated (UT)) for 24 hours.

FIG. 6D is a graph comparing the activation of HEK-Blue cells by MnO nanoparticles, MnO@Curcumin nanoparticles, and curcumin at different mass ratios with respect to CpG, in the presence of FBS for 24 hours.

FIG. 6E is a graph comparing the activation of hTLR9 cells by MnO nanoparticles, MnO@Curcumin nanoparticles, and curcumin at different mass ratios with respect to poly (I:C), in the presence of FBS for 24 hours.

FIG. 6F is a graph comparing activation of hTLR3 cells by MnO nanoparticles, MnO@Curcumin nanoparticles, and curcumin at different mass ratios with respect to LPS, in the presence of FBS for 24 hours.

FIG. 6G shows the inhibition of CpG-activation, comparing untreated (UT) cells against cells treated with CpG, CpG and curcumin, CpG and MnO nanoparticles, and CpG and MnO@Curcumin nanoparticles, evaluated using a transwell assay.

FIG. 6H shows LPS-activated macrophage-induced macrophage migration, comparing untreated (UT) cells against cells treated with LPS, LPS and curcumin, LPS and MnO nanoparticles, and LPS and MnO@Curcumin nanoparticles, evaluated using a transwell assay.

FIG. 7A is a schematic illustration of a transwell-Matrigel invasion assay.

FIG. 7B shows the invasion of MDA-MB-231 cells treated with a control formulation, DAMPs, and DAMPs with MnO nanoparticles.

FIG. 7C is a schematic illustration of a wound healing assay.

FIG. 7D shows wound healing images of MDA-MB-231 cells treated with a control medium, DAMPs, and DAMPs with MnO nanoparticles for varying time periods.

FIG. 7E is a graph comparing migration of MDA-MB-231 cells treated with a control medium, DAMPs, and DAMPs with MnO nanoparticles.

FIG. 8A is a graph showing the fluorescent intensity of 4T1 tumor-bearing living mice at differing times after intratumor injection of MnO@Curcumin@IR780 nanoparticles.

FIG. 8B shows representative fluorescent images of the 4T1 tumor-bearing living mice at differing times after the intratumor injection of MnO@Curcumin@IR780 nanoparticles.

FIG. 8C shows an ex vivo fluorescent image of excised major organs and tumors from mice after injection with MnO@Curcumin@IR780 nanoparticle solution for 6 hours.

FIG. 9A is a graph comparing body weight of mice treated with PBS, MnO nanoparticles, curcumin, MnO@Curcumin nanoparticles, laser treatment, laser treatment combined with curcumin, laser treatment combined with MnO nanoparticles, and laser treatment combined with MnO@Curcumin nanoparticles.

FIG. 9B is a series of graphs comparing tumor volume of mice treated with PBS, MnO nanoparticles, curcumin, MnO@Curcumin nanoparticles, laser treatment, laser treatment combined with curcumin, laser treatment combined with MnO nanoparticles, and laser treatment combined with MnO@Curcumin nanoparticles.

FIG. 9C shows IVIS® images of mice treated with PBS, MnO nanoparticles, curcumin, MnO@Curcumin nanoparticles, laser treatment, laser treatment combined with curcumin, laser treatment combined with MnO nanoparticles, and laser treatment combined with MnO@Curcumin nanoparticles.

FIG. 9D shows images of excised tumors from the mice treated with PBS, MnO nanoparticles, curcumin, MnO@Curcumin nanoparticles, laser treatment, laser treatment combined with curcumin, laser treatment combined with MnO nanoparticles, and laser treatment combined with MnO@Curcumin nanoparticles.

FIG. 9E is a graph comparing the survival rates of the mice treated with PBS, MnO nanoparticles, curcumin, MnO@Curcumin nanoparticles, laser treatment, laser treatment combined with curcumin, laser treatment combined with MnO nanoparticles, and laser treatment combined with MnO@Curcumin nanoparticles.

FIG. 10 shows immune cell subset profiling in the blood of recovered versus deceased COVID-19 patients in the intensive care unit (ICU) with and without dexamethasone. Data for immune cell subsets are presented as a percentage of total leukocytes (TL) or total monocytes (TM) on the last day of study. The shaded areas highlight the normal range for each immune cell subset. A. CD3⁺ T cell percentages are consistent with lymphopenia regardless of clinical status; (+) dexamethasone trends lower than (−) dexamethasone. B. CD4⁺ T cell percentages are consistent with total T cells. C. CD8⁺ T cell percentages are consistent with CD4⁺ T cells and total T cells. D. Natural killer (NK) cell percentages are below normal range regardless of clinical or dexamethasone status. E. Natural killer T (NKT) cell percentages are higher than normal in (−) dexamethasone patients regardless of clinical status. (+) dexamethasone patients have NKT cell percentages closer to the normal range with discharged patients trending higher than deceased patients. F. B cell percentages are below normal ranges regardless of clinical or dexamethasone status. G. Neutrophil percentages are above normal ranges regardless of clinical or dexamethasone status. H. Amongst recovered patients, eosinophils are higher than normal in (−) dexamethasone and within the normal range in (+) dexamethasone; this pattern is reversed amongst deceased patients. I. Basophil percentages are within normal range regardless of clinical or dexamethasone status. J. Total monocyte percentages are within normal range regardless of clinical or dexamethasone status. K. CD16⁻ monocyte percentages trend lower in (−) dexamethasone patients that were discharged versus those that died while CD16⁻ monocyte percentages were roughly equivalent in (+) dexamethasone patients regardless of clinical outcome. L. CD16⁺ monocyte percentages are significantly higher in (−) dexamethasone patients that were discharged versus those that died while CD16⁺ monocyte percentages were roughly equivalent in (+) dexamethasone patients regardless of clinical outcome. *=p<0.05, ns=not significant via 2-tailed t-test; no statistically significant differences were noticed unless indicated in the figure.

FIG. 11 shows longitudinal Toll-like receptor (TLR) activation profiling of serum and endotracheal aspirate (ETA) from ICU patients with COVID-19 using HEK-TLR reporter cells for TLRs 3, 4, 7, and 9. A. Longitudinal profiling of TLR3, TLR4, TLR7, and TLR9 activation using COVID-19 patient serum shows the following: (1) strong activation of TLR3 and TLR7 by serum across all timepoints; (2) activation of TLR4 by serum on days 1, 3, 14, and 21; and (3) activation of TLR9 by serum on days 3, 14, and 21. B. COVID-19 patient serum and ETA significantly activate TLR3, TLR4, TLR7, and TLR9 compared to normal serum. *=p<0.05 via 2-tailed t-test; ***=p<0.001 via 2-tailed t-test; ****=p<0.0001 via 2-tailed t-test; ns=not significant via 2-tailed t-test; D=day; dil=dilution.

FIG. 12 shows that treating serum and ETA from COVID-19 ICU patients with nucleic acid-binding microfibers reduces the activity and quantity of TLR activating damage and pathogen associated molecular patterns (DAMPs/PAMPs). Treatment of serum with nucleic acid-binding fiber significantly reduces TLR activation of HEK-TLR reporter cells for A. TLR3, B. TLR4, C. TLR7, and D. TLR9. Treatment of ETA with nucleic acid-binding fiber significantly reduces TLR activation of HEK-TLR reporter cells for E. TLR3, F. TLR4, G. TLR7, and H. TLR9. Treatment of serum with nucleic acid-binding fiber significantly reduces levels of I. DNA; J. RNA; K. nucleosome; and L. HMGB-1. Treatment of ETA with nucleic acid-binding fiber significantly reduces levels of M. DNA; N. RNA; O. nucleosome; and P. HMGB-1. Q. Treatment of serum with nucleic acid-binding fiber modestly reduces albumin levels. R. Treatment of ETA with nucleic acid-binding fiber has no effect on albumin levels. *=p<0.05 via 2-tailed t-test; ***=p<0.001 via 2-tailed t-test; ****=p<0.0001 via 2-tailed t-test; NS=not significant via 2-tailed t-test.

FIG. 13 shows myeloid activation and PAMP carrier consumption in deceased patients using proteomics. A. Significant pathway (adjusted p<0.05) similarity clustering of differentially upregulated proteome in deceased patients relative to survivors. B. High confidence (>0.9 confidence) STRING network of myeloid related immune interactions of differentially upregulated proteome and their dominating pathways C. Expression heatmap for immune-related differentially upregulated (red) and downregulated (blue) proteins over time relative to the average value for all infected patients, ordered by their levels compared to uninfected controls (right bar). D. High confidence (>0.9 confidence) STRING network for protein interactions of differentially downregulated proteome and dominating pathways. E. Time-series plots for differentially expressed protein hits with trending and significant timepoint values.

FIG. 14 shows that manganese oxide (MnO) nanoparticles inhibit agonist mediated TLR activation. MnO nanoparticles inhibit activation of HEK-Blue hTLR3 (left), hTLR8 (middle), and hTLR9 (right) cells in the absence or presence of fetal bovine serum (FBS) after nanomaterial treatment for 24 hours in a dose-dependent manner. Experiments were repeated (N=3). Data are presented as bars ±SEM. All agonists were purchased from Invivogen. ORN refers to ORN06/LyoVec.

FIG. 15 shows that MnO nanoparticles reduce the activity of TLR activating PAMPs/DAMPs in COVID ETA and serum and prevent DAMP/PAMP-mediated TLR tolerance in monocytes. MnO nanoparticles significantly reduce stimulation of TLRs 3, 4, 7, and 9 by COVID-19 serum in TLR reporter cell assays. B. MnO nanoparticles significantly reduce stimulation of TLRs 3, 4, 7, and 9 by COVID-19 ETA in TLR reporter cell assays. C. Freshly isolated monocytes were treated with or without polyinosinic:polycytidylic acid (poly I:C, a double-stranded viral RNA DAMP mimic) for 24 hours, followed by treatment with or without lipopolysaccharide (LPS, a bacterial DAMP mimic) for 5 hours, after which cell supernatant was collected and IL-6 levels were quantified. Treatment with MnO nanoparticles significantly reduced IL-6 production by monocytes in response to sequential TLR stimulation. **=p<0.01 via 2-tailed t-test; ****=p<0.0001 via 2-tailed t-test.

FIG. 16 shows a longitudinal characterization of proteomic analysis from recovered versus deceased patients. A. Heatmap of all differentially upregulated (red), and downregulated (blue) proteins between deceased and survived patients for timepoints measured relative to the average value between both outcomes (left of protein labels) and ordered by the differential expression of deceased samples relative to healthy individuals (right). B. Time course plots for explored differentially expressed proteins. C. Boxplots for individual sample protein intensity distributions before and after normalization (see methods). D. Proteomics profiled patient cohort demographics.

FIG. 17 shows that the MnO nanoparticles reduce plaque formation of multiple strains of SARS-CoV-2, i.e., the European ‘G’ variant (left) and the South African ‘B.1.1.7’ variant (right).

### DETAILED DESCRIPTION

To better understand the pathology of COVID-19, the present inventors studied blood and respiratory samples from COVID-19 intensive care unit (ICU) patients. They found that these patients suffered from an inflammatory storm stimulated by circulating damage-associated molecular patterns (DAMPs) and pathogen-associated molecular patterns (PAMPs) released by dead and dying cells. They observed that prolonged, high levels of DAMPs/PAMPs hyperstimulate monocytes, leading to their exhaustion (i.e., immune tolerance) in COVID-19 patients that succumb to the disease. Finally, they showed that a manganese oxide (MnO) nanoparticle can be used to limit the ability of DAMPs/PAMPs to induce monocyte tolerance. These nanoparticles have potential for use as therapeutics that combat hyperinflammation and immune exhaustion in patients with severe COVID-19 as well as other diseases. These nanoparticles offer several advantages over the available treatments for SARS-CoV-2, including (1) that they can be used against any SARS-CoV-2 variant without modification to adjust for mutations, and (2) that they can be stored at 4° C., which makes them more accessible than vaccines that require storage at −80° C. Importantly, they are also biodegradable and have low cytotoxicity.

### Compositions

The main component of the MnO nanoparticles is manganese oxide, which may be synthesized by combining a manganese compound and an acid at high temperature. Manganese oxide synthesis may be performed by mixing the manganese compound and the acid in a solvent for at least one minute to form a mixture, which is then stirred, heated at a temperature of about 90-175° C. for at least 1 hour, and then allowed to cool naturally. Anionic manganese oxide nanoparticles are then extracted from the cooled mixture. For example, the inventors prepared their MnO nanoparticles by mixing manganese acetate and tannic acid at a mass ratio of 1:2-6 in Milli-Q® ultrapurified water for 10 minutes at room temperature, heating the mixture at 150° C. for 2 hours, and then cooling it to <50° C. naturally.

Suitable manganese compounds for making manganese oxide include, but are not limited to, manganese phosphate, manganese oxide, manganese acetate, manganese sulfide, manganese dioxide, manganese heptoxide, manganese chloride, manganese carbonate, and the like. The nucleic-acid scavenging activity of the MnO nanoparticles is derived from the acid, which acts as a surface ligand. Thus, suitable acids include phenolic acids that have the ability to bind to DNA, such as tannic acid, ellagic acid, gallic acid, and other derivatives of tannic acid. Suitable solvents include water, ethanol, methanol, and mixtures of water with ethanol or methanol. In certain embodiments, the manganese compound is manganese acetate, the acid is tannic acid, and the solvent is water.

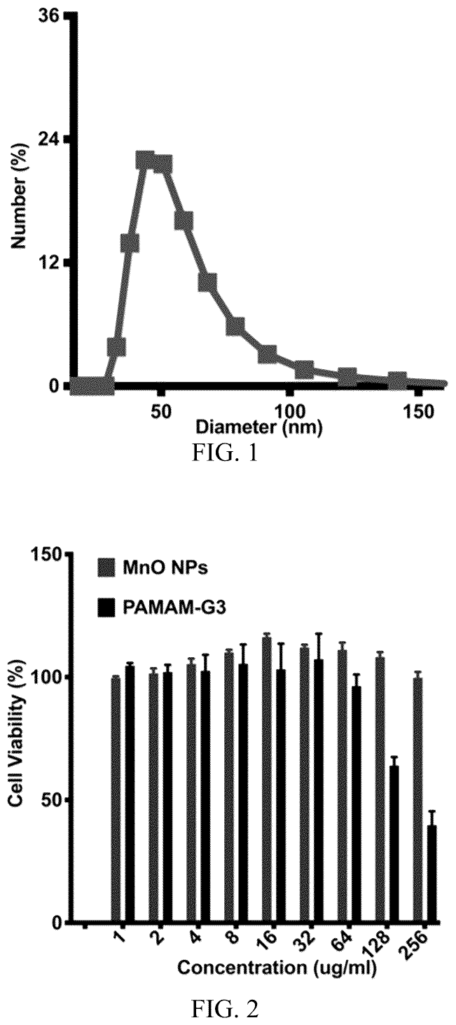

The MnO nanoparticles may have a size ranging between approximately 5 nm and 125 nm, a mass ratio of manganese compound to acid ranging between about 0.5:1-7 and 1:2-6, and/or a zeta potential ranging between about −5 mV and −30 mV. For example, the MnO nanoparticles generated by the inventors have a size of about 30 to 100 nm (FIG. 1) and have a zeta potential of about −20 mV.

As will be discussed in greater detail below, the inventors characterized the anti-inflammatory therapeutic activity of the MnO nanoparticles in vitro and in vivo, with a focus on three mechanisms of action: (1) binding and scavenging inflammatory cell-free DNA (cfDNA), which inhibits cfDNA-induced TLR activation and nuclear factor kappa B (NF-kB) signaling; (2) inhibition of activated macrophage-induced macrophage recruitment; and (3) inhibition of tumor cell migration and invasion.

Researchers have demonstrated that soluble cationic nucleic acid-binding polymers (NABPs) and NABP-based nanoparticles can be used to decrease TLR9 activation by scavenging cfDNA and associated lipid/protein complexes. However, amine-terminated dendrimers were recently shown to activate platelets and cause a fatal, disseminated intravascular coagulation (DIC)-like condition in mice and rats. Thus, the potential cytotoxicity of NABPs may limit their clinical use. In FIG. 2, the inventors demonstrate that the MnO nanoparticles are less cytotoxic than the nucleic acid-binding cationic polymer PAMAM-G3 (FIG. 2). Cytotoxicity was evaluated using an MTS assay. To perform this assay, RAW264.7 cells were plated into a 96-well plate at a density of 1×10⁴ cells/well and were allowed to adhere overnight in growth medium at 37° C., 5% CO2. The cells were treated with MnO nanoparticles or PAMAM-G3 at various concentrations for 24 hours. Then, the medium was replaced with 100 μL medium containing 10% MTS, and cell viability was determined using a multiwell plate reader to measure the OD at 490 nm.

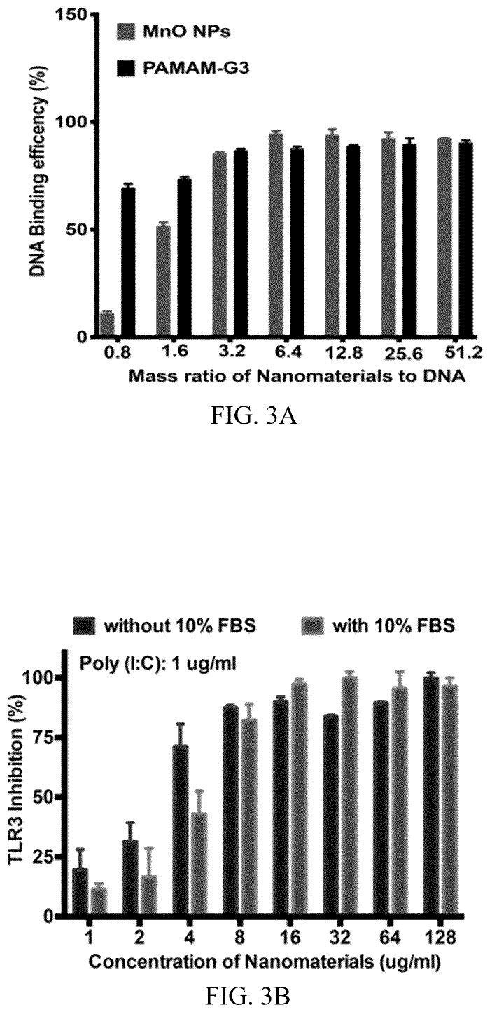

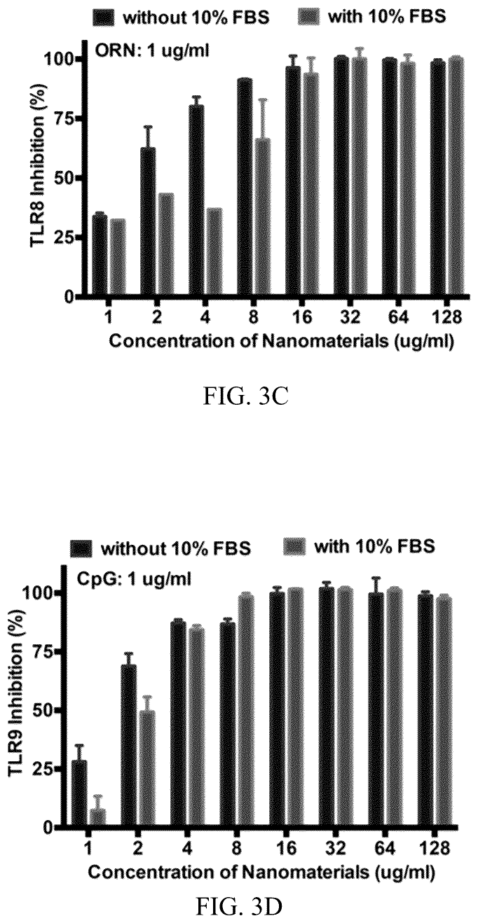

In FIG. 3A, the inventors demonstrate that the MnO nanoparticles have a strong affinity for DNA. This data was generated using a DNA-binding assay in which 12.5 μL Quant-iT PicoGreen® and 25 μL 5 mg/mL calf thymus DNA were mixed with 1×Tris-EDTA (TE) buffer in the dark. 100 μL of different concentrations of MnO nanoparticles were combined with 100 μL of the above solution and were added to a 96-well black plate and incubated at 37° C. for 30 minutes. The fluorescence intensity of the unbound Picogreen®-DNA complex at 520 nm was measured with a multiwell plate reader via excitation at 490 nm. The inventors found that, despite their net negative surface charge, the MnO nanoparticles exhibit high DNA binding affinity, which is comparable to that of the commercially available scavenger polymer PAMAM-G3.

In FIG. 3B-D, the inventors demonstrate that the MnO nanoparticles inhibit agonist-induced activation of Toll-like receptors (TLRs). HEK-Blue hTLR3, hTLR8, and hTLR9 cells were obtained by co-transfecting the hTLR gene and an optimized secreted embryonic alkaline phosphatase (SEAP) reporter gene into HEK293 cells. HEK-Blue hTLR cells were cultured and maintained in DMEM with 10% FBS and 1% penicillin-streptomycin. To evaluate the ability of the MnO nanoparticles to inhibit TLR activation, HEK-Blue hTLR cells were seeded in a 96-well plate for 1 hour and then treated with 2 μL of agonist (CPG Bw006 for TLR9, Poly (I:C) for TLR3, or ORN06/Lyo Vec for TLR8, 1 mg/mL). After 10 minutes of incubation, 20 μL of MnO nanoparticles at different concentrations were introduced in a final volume of 200 μL. After 24 hours, the supernatants were collected and mixed with Quanti-Blue™. TLR activation associated with SEAP activity was determined with a multiwell plate reader by measuring the OD at 620 nm. The cell densities (in a 96 well plate) used were 8×10⁴ cells/well for HEK-Blue hTLR9 cells, 5×10⁴ cells/well for HEK-Blue hTLR3 cells, and 4×10⁴ cells/well for HEK-Blue hTLR8 cells. The inventors found that the MnO nanoparticles inhibit CpG-induced activation of HEK-Blue hTLR9 cells, inhibit poly (I:C)-induced activation of HEK-Blue hTLR3 cells, and inhibit ORN06/Lyo Vec (ORN)-induced activation of HEK-Blue hTLR8 cells in a dose-dependent manner, regardless of the presence or absence of FBS.

The MnO nanoparticles can be used to mitigate inflammatory responses, such as the inflammatory response caused by photothermal therapy (PTT). PTT is a method for treating medical conditions that utilizes near-infrared (NIR) light-absorbing agents to convert photoenergy into heat for thermal ablation. It is regarded as a minimally invasive and highly efficient treatment for cancer. Owing to the good controllability of near-IR light and the negligible toxicity of photothermal agents (PTAs) in the dark, PTT can eliminate tumor cells specifically without harming normal tissues. However, because of the hyperthermia induced by PTAs, the most possible cellular death mode after PTT is necrosis, which is characterized by rupture of the plasma membrane and the subsequent release of cellular contents. The DAMPs and PAMPs released during necrosis initiate inflammatory reactions that can cause a cytokine storm, multiple organ failure, and death. Inflammatory circulating cfDNA, i.e., nuclear or mitochondrial DNA released by damaged host cells, is one such DAMP. Inflammation induced by PTT may cause severe adverse effects, including tumor regeneration, metastatic dissemination, and therapeutic resistance. Therefore, effective alleviation of inflammatory responses caused by PTT is of great significance for tumor treatment.

To increase the anti-inflammatory effects of the MnO nanoparticles, they may be combined with curcumin, forming what are hereinafter referred to as “MnO@Curcumin” nanoparticles. Curcumin is a natural anti-inflammatory agent that has been used to treat medical conditions for many years. The MnO@Curcumin nanoparticles may be decorated with IR780 iodide, a near-infrared (NIR) dye, to form “MnO@Curcumin@IR780” nanoparticles. IR780 iodide can be used to simultaneously achieve PTT and photodynamic therapy (PDT) effects in the presence of laser irradiation and has been approved by FDA for clinical applications. However, the hydrophobic nature and the lack of tumor specificity of such photosensitizers limit their clinical applications. Thus, the inventors utilized MnO@Curcumin nanoparticles, which have excellent solubility, as carriers for IR780 iodide. MnO@Curcumin@IR780 nanoparticles can be used for PTT, as a pathogenic cfDNA scavenger, and as anti-inflammatory agents.

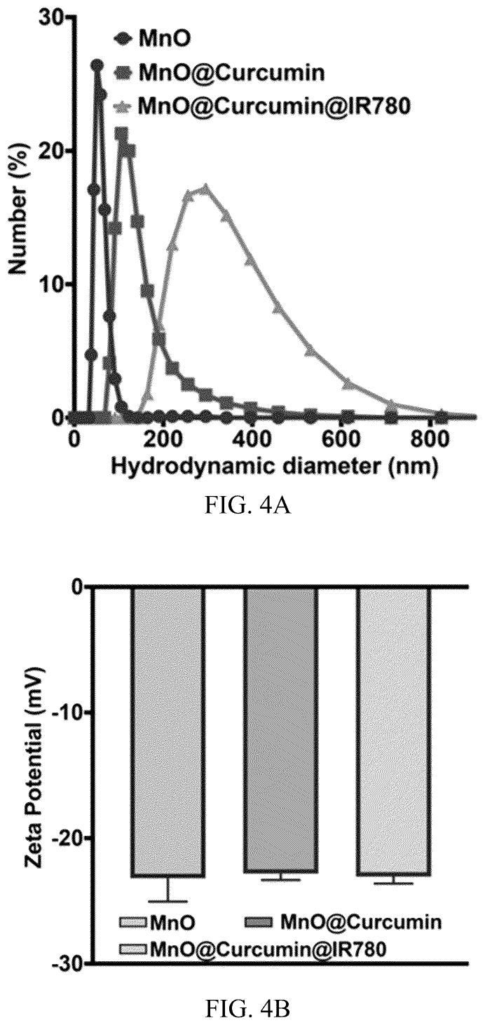

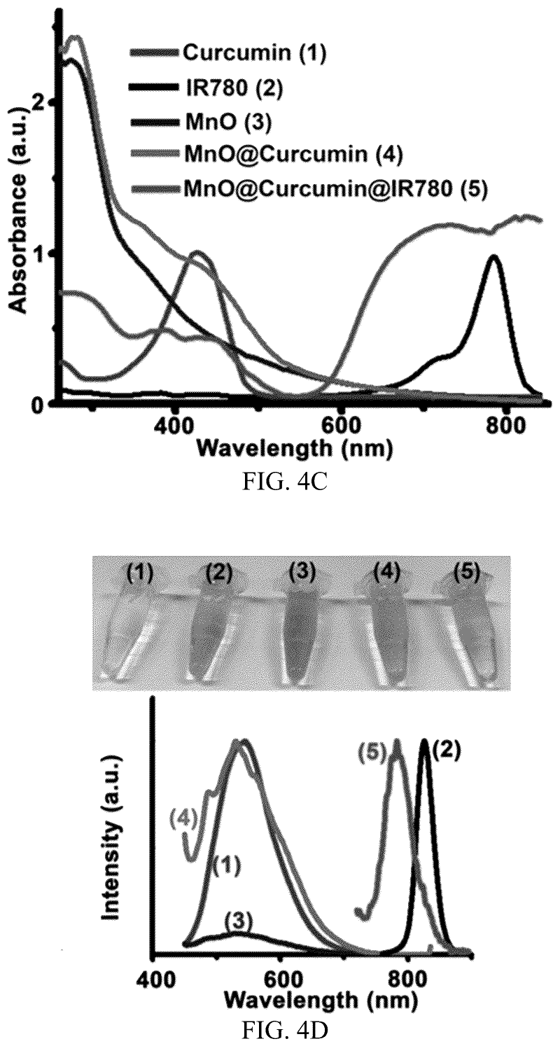

As shown in FIG. 4A, the MnO@Curcumin nanoparticles exhibited a larger hydrodynamic size (˜100 nm) than the MnO nanoparticles (˜50 nm), and the MnO@Curcumin@IR780 nanoparticles were even larger (˜240 nm). The MnO, MnO@Curcumin, and MnO@Curcumin@IR780 nanoparticles all displayed negatively charged surfaces, and a zeta potential of about −23 mV (FIG. 4B). UV-vis absorption spectra, photoluminescence (PL) spectra, and photographs of the MnO, MnO@Curcumin, and MnO@Curcumin@IR780 nanoparticles are shown in FIGS. 4C and 4D. The absorption spectra of the MnO@Curcumin nanoparticles show an obvious peak at ˜430 nm compared with the MnO nanoparticles, which is ascribed to the curcumin. Moreover, the PL spectra of MnO@Curcumin contains the characteristic PL peak of curcumin at λₘₐₓ of ˜550 nm (FIG. 4D). MnO@Curcumin@IR780 nanoparticles exhibited a broad absorption band (˜600-800 nm) and an obvious PL peak at ˜780 nm, which was attributed to IR780, demonstrating the successful decoration of IR780 in the MnO@Curcumin nanoparticles. The increased hydrodynamic size of the MnO@Curcumin@IR780 nanoparticles relative to the MnO@Curcumin nanoparticles suggests successful functionalization with curcumin (IR780), which was further evidenced by color change, and a changed absorption/PL spectra.

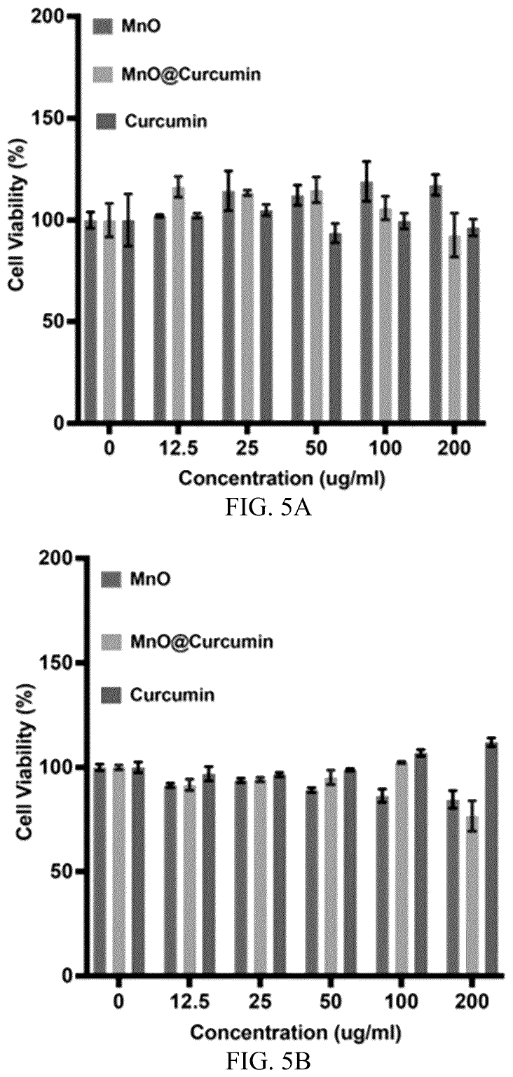

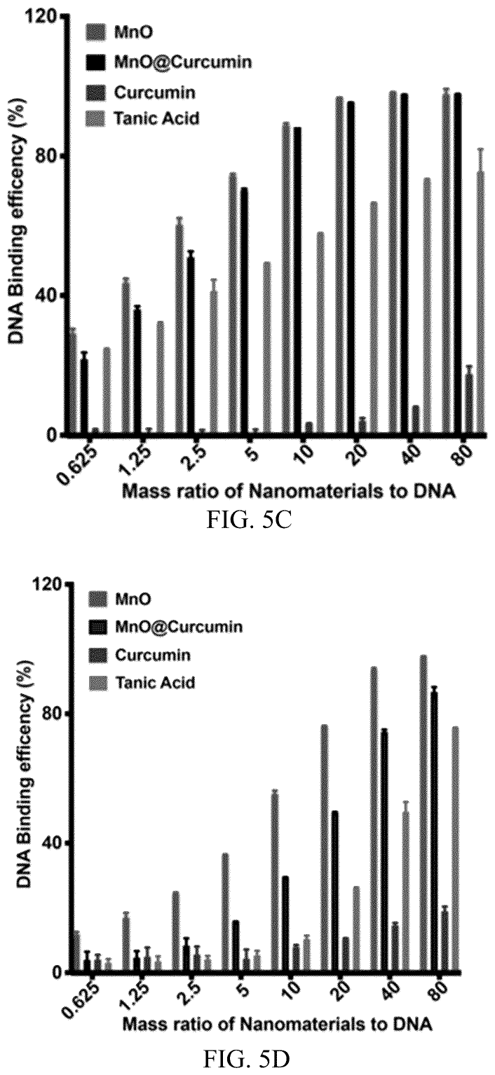

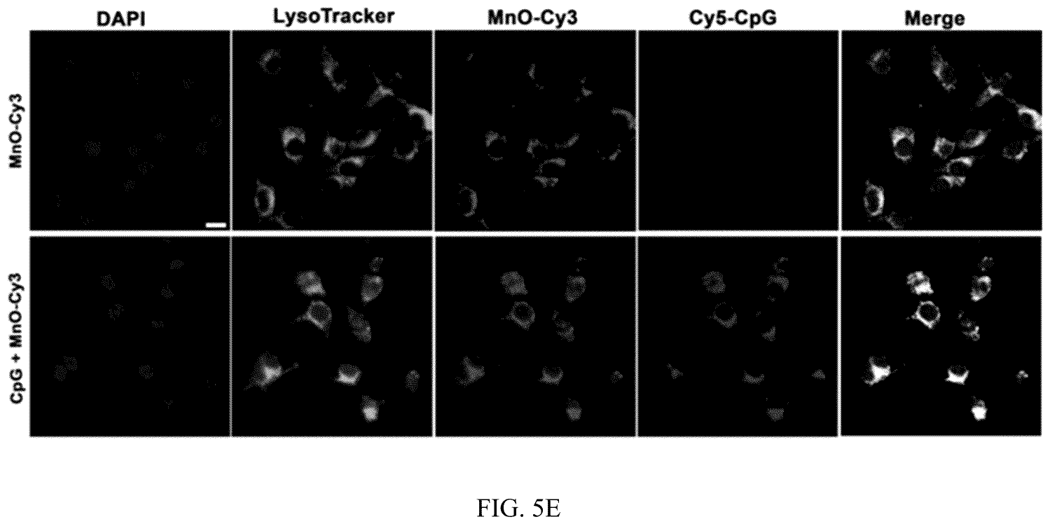

To evaluate the ability of MnO and MnO@Curcumin nanoparticles to mitigate anti-PTT-induced inflammation, the inventors first investigated their cytotoxicity in vitro by MTS assay. The results revealed that MnO and MnO@Curcumin nanoparticles exhibited negligible cytotoxicity against RAW264.7 murine macrophage cells (FIG. 5A) and 4T1 cells (FIG. 5B). In addition, the cytotoxicity of MnO@Curcumin@IR780 nanoparticles to 4T1 cells in the presence and absence of NIR laser irradiation also was studied.

Inflammatory circulating cfDNA released by PTT-damaged host cells might cause a severe inflammatory response, which may be mitigated by the scavenging of cfDNA. Therefore, to interrogate the cfDNA scavenging ability of the various nanoparticles, the inventors investigated the calf thymus DNA binding affinity of the nanoparticles in Tris-EDTA (TE) buffer with and without 10% FBS by measuring unbound PicoGreen®-labeled DNA (FIGS. 5C and 5D). As shown in FIGS. 5C and 5D, in contrast to negligible DNA binding ability of curcumin, free tannic acid can bind to DNA, which is consistent with previous reports. Significantly, MnO and MnO@Curcumin nanoparticles exhibited high DNA binding affinity despite their net negative surface charge, which is attributed to their tannic acid functional ligands. Interestingly, the MnO and MnO@Curcumin nanoparticles displayed stronger DNA binding ability than free tannic acid, possibly due to their nanostructures with high specific surface area. The DNA binding ability was reduced with the addition of 10% FBS in all groups, due to competitive interactions of serum proteins, but this effect was overcome by increasing the mass ratio of nanomaterials to DNA. These results suggest that MnO and MnO@Curcumin nanoparticles can act as scavengers that bind cfDNA released by apoptotic and necrotic cancer cells.

cfDNA can activate an inflammatory response via TLR activation. Specifically, unmethylated, CpG motif-bearing cfDNA species (referred to herein as “CpG DNA”) can activate TLR9. TLR9 initiates a signaling cascade involving nuclear factor-kappa B (NF-kB) that culminates in the up-regulation of pro-inflammatory pathways. Therefore, the inventors internalized the nanoparticles and assessed their ability to block cfDNA-TLR interactions in vitro. FIG. 5E (top) demonstrates that MnO-Cy3 nanoparticles were internalized by RAW264.7 cells and localized in endolysosomes, demonstrating high cellular uptake efficiency of nanoparticles. Colocalization of Cy5-CpG and MnO-Cy3 fluorescent signals was observed in the endolysosomal compartment (bottom of FIG. 5E), indicating that nanoparticles could enter the cells and scavenge the cfDNA in the endolysosomal compartment.

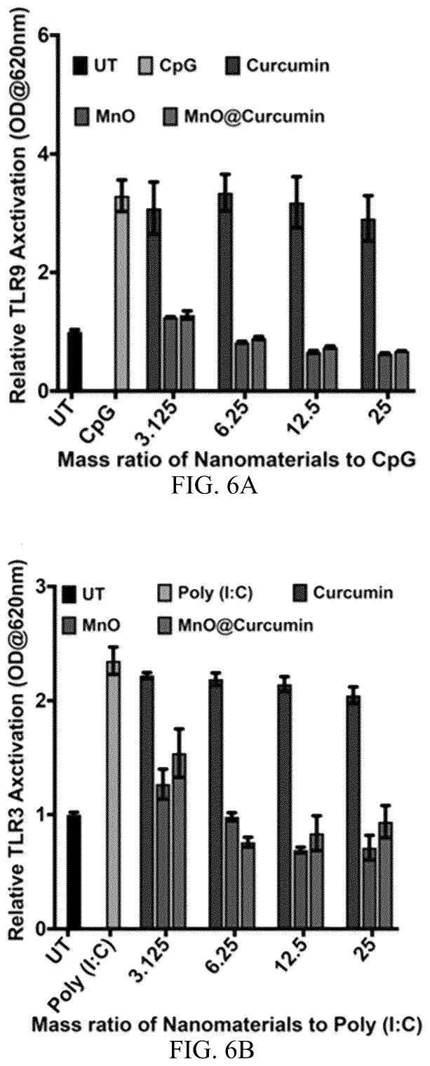

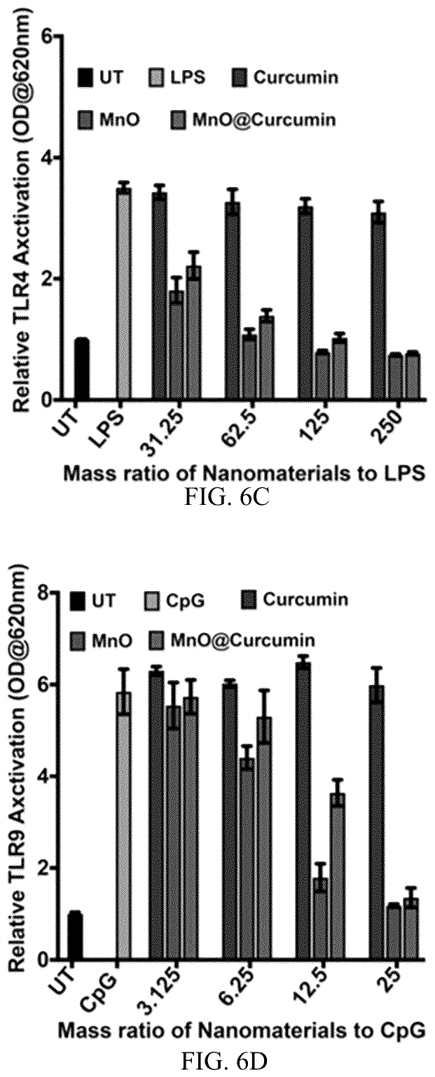

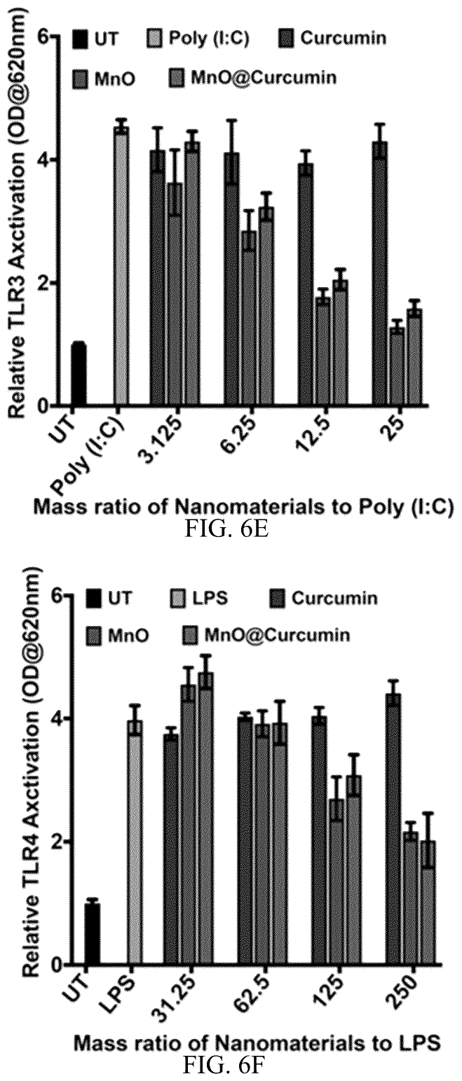

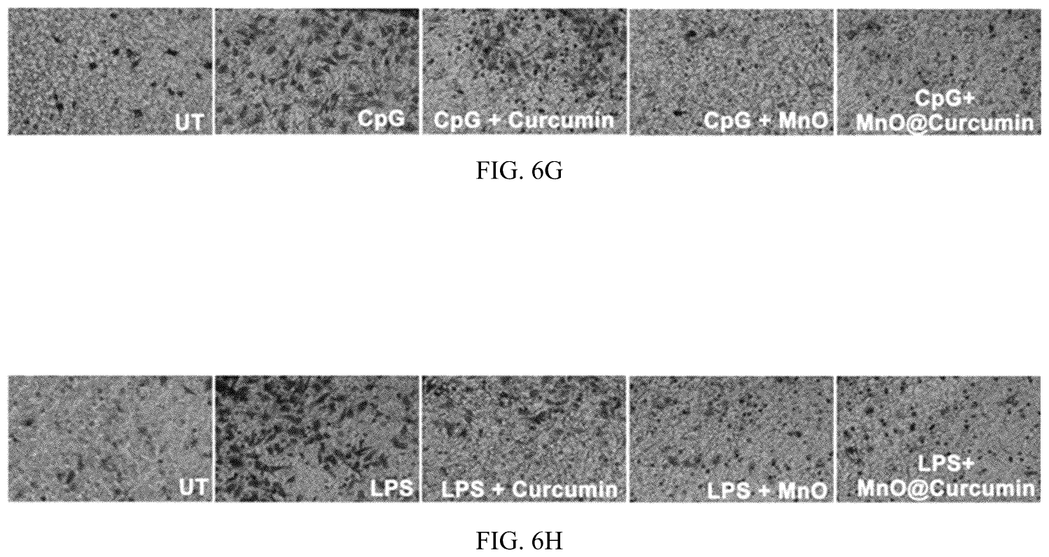

The inventors also studied the ability of the nanoparticles to inhibit nucleic acid-induced TLR activation using HEK-Blue human TLR (hTLR) cells. The MnO and MnO@Curcumin nanoparticles can inhibit CpG-induced activation of HEK-Blue hTLR9 cells, poly (I:C)-induced activation of HEK-Blue hTLR3 cells, as well as LPS-induced activation of HEK-Blue hTLR4 cells in a dose-dependent manner, regardless of the presence or absence of FBS (FIGS. 6A-6F). In contrast, curcumin cannot inhibit agonist-induced activation of HEK-Blue hTLR9/3/4. These results indicate that the MnO and MnO@Curcumin nanoparticles not only inhibit cfDNA and cfRNA, but also inhibit LPS-induced TLR activation. Macrophage accumulation in inflamed sites exacerbates inflammation. Reducing activated macrophage-induced macrophage recruitment is a promising strategy for alleviating PTT-induced inflammation in photothermal cancer therapy. As shown in FIGS. 6G and 6H, attractants released by CpG Bw006- and LPS-activated RAW264.7 macrophages induced chemotaxis, which led to the recruitment of macrophages from the upper side of the transwell chamber to the lower side. The addition of MnO and MnO@Curcumin nanoparticles was shown to largely reduce macrophage migration, demonstrating that the MnO and MnO@Curcumin nanoparticles can inhibit activated macrophage-induce macrophages migration once macrophages are activated.

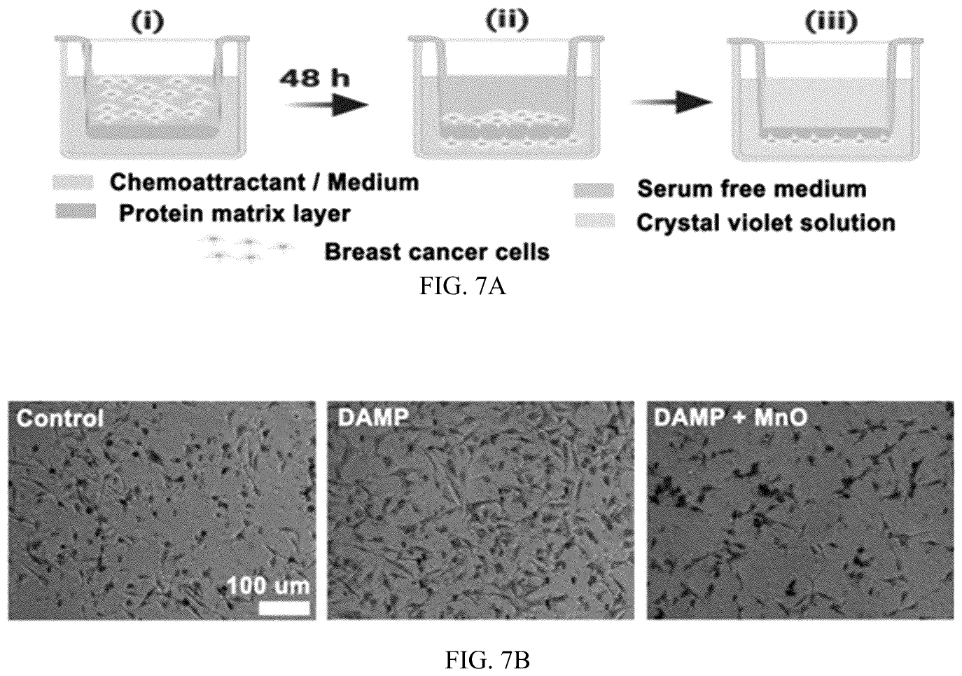

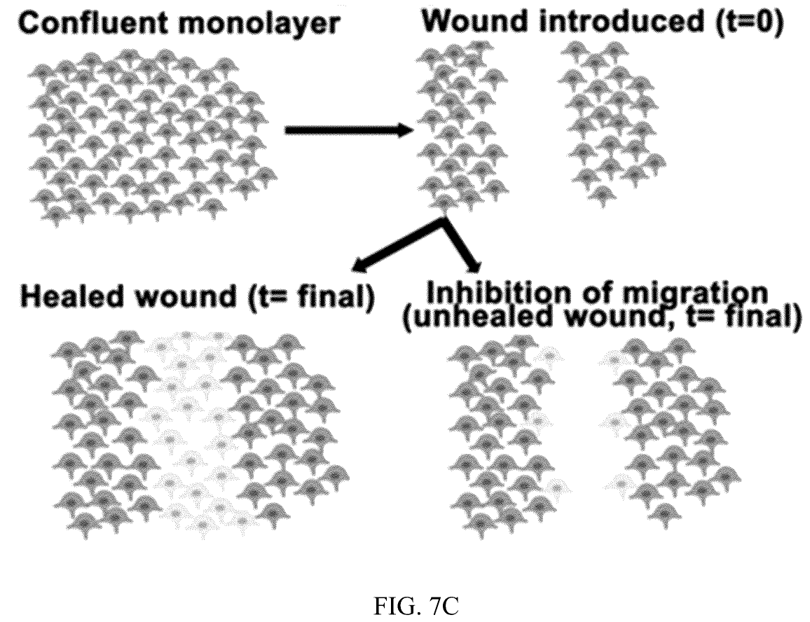

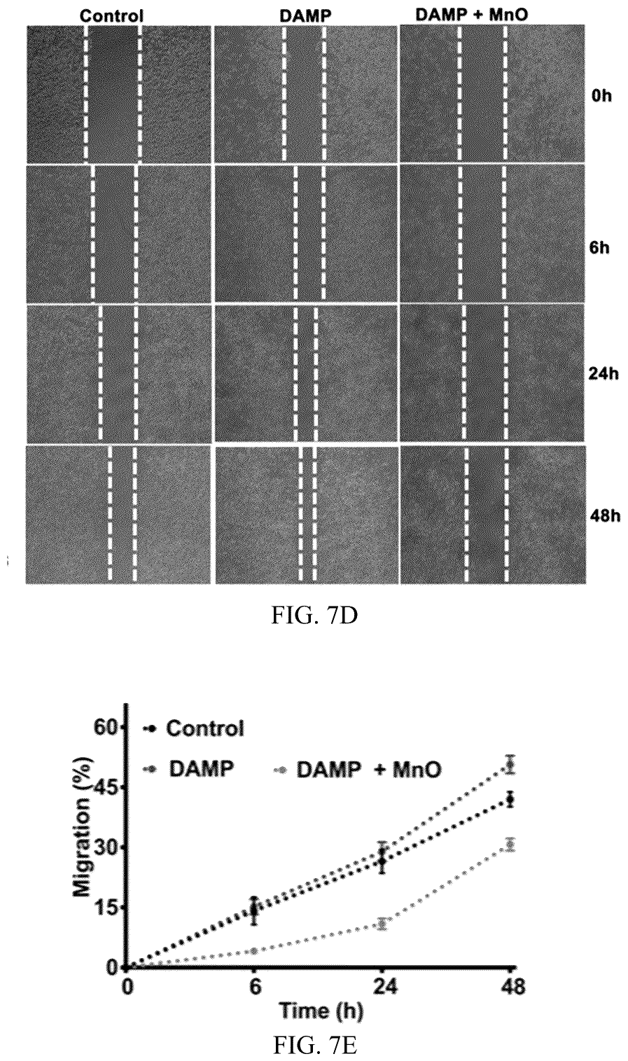

Most cancer deaths are caused by metastases wherein tumor cells migrate and invade other tissues. Thus, the inventors evaluated whether MnO and MnO@Curcumin can inhibit breast cancer cell invasion and migration using transwell-Matrigel invasion assays, as shown in FIGS. 7A-7E. Here, the addition of DAMPs of MDA-MB-231 cells, prepared by a freeze-thaw method, induced MDA-MB-231 tumor cell invasion, and these effects were significantly inhibited by MnO nanoparticle treatment (FIGS. 7A and 7B). A schematic illustration of these results is shown in FIG. 7C, pictures are shown in FIG. 7D, and a quantification is shown in FIG. 7E. After 48 hours of treatment, the wound-healing areas of the group with DAMP rapidly recovered and almost completely closed with time (FIG. 7E). In contrast, the wound repair was obviously impeded after treatment with MnO nanoparticles. These findings suggest that MnO can neutralize the in vitro pro-invasive effects and migration of DAMPs.

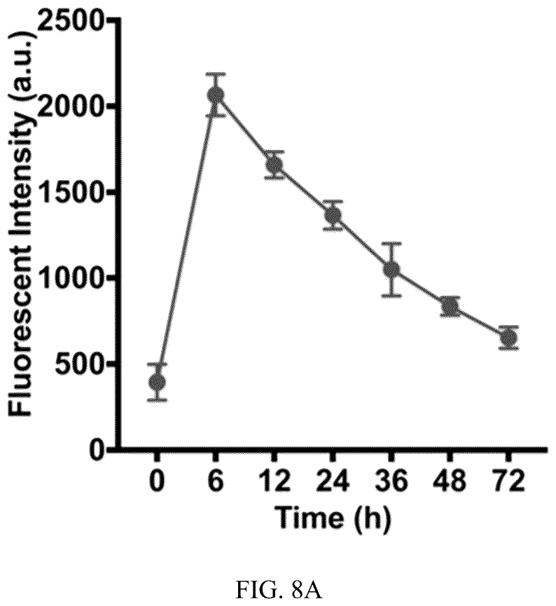

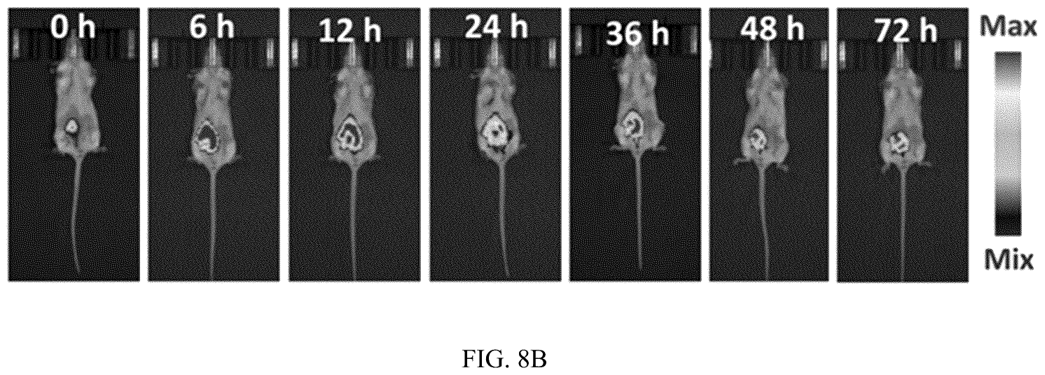

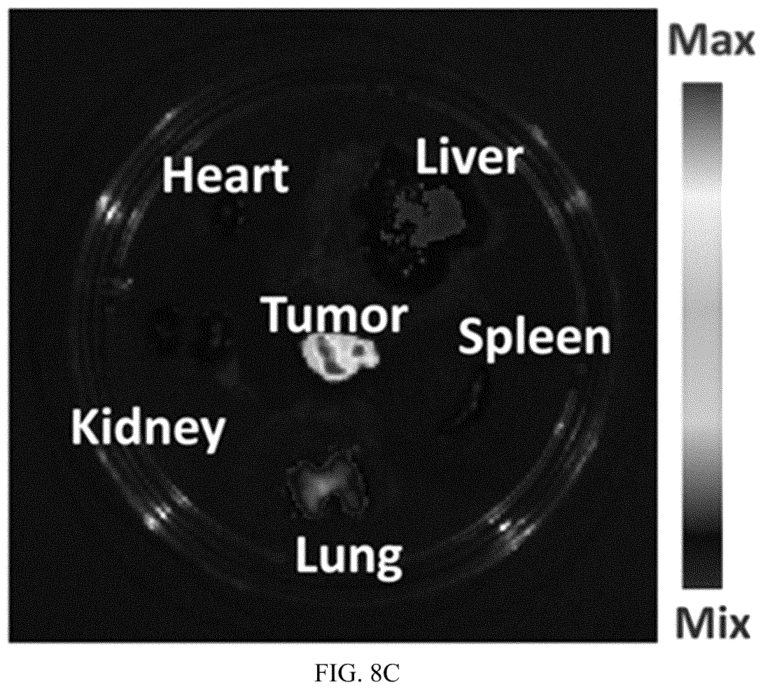

To investigate the effect of MnO and MnO@Curcumin on the inflammatory response caused by PTT, the inventors established a 4T1 metastasis tumor model by inoculating 4T1-Luciferase cells into the subcutaneous fat pad of Balb/c mice. When the tumor volume was 80-100 mm³, 4T1 tumor-bearing mice were injected intratumorally with MnO@Curcumin@IR780 nanoparticles to conduct near-infrared fluorescent photography and phototherapy. The fluorescent signal intensity was recorded on the IVIS® instrument at different time intervals. As observed in FIG. 8A, the fluorescent signal of the tumor site reached the highest level at 6 hours post-injection and then decreased with time, which was consistent with the fluorescent images of FIG. 8B. Thus, after 6 hours post-injection, the mice were irradiated with an 808 nm laser (1 W/cm²) for 10 minutes to investigate biodistribution and tumor accumulation behavior of MnO@Curcumin@IR780 nanoparticles. Ex vivo fluorescence imaging of 4T1 tumor-bearing mice was carried out using MnO@Curcumin@IR780 nanoparticles. As shown in FIG. 8C, there was a strong fluoresce signal at the tumor region, demonstrating efficient tumor accumulation of MnO@Curcumin@IR780 nanoparticles. Additionally, the liver and lung also displayed visible dim fluorescence, which could be due to the absorption of the mononuclear phagocyte system.

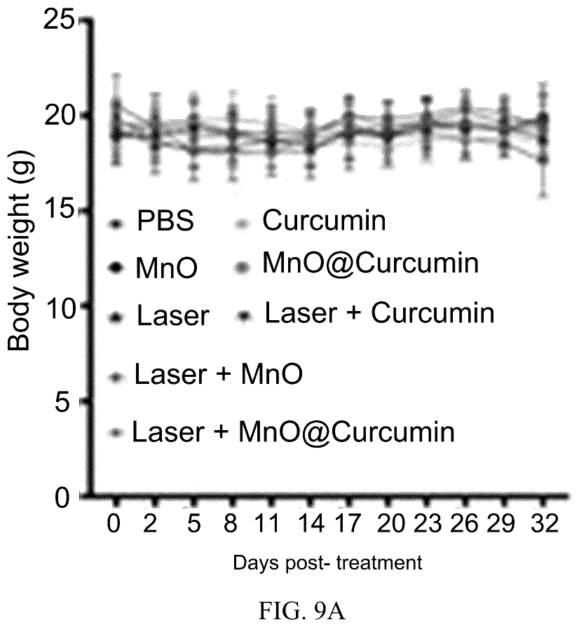

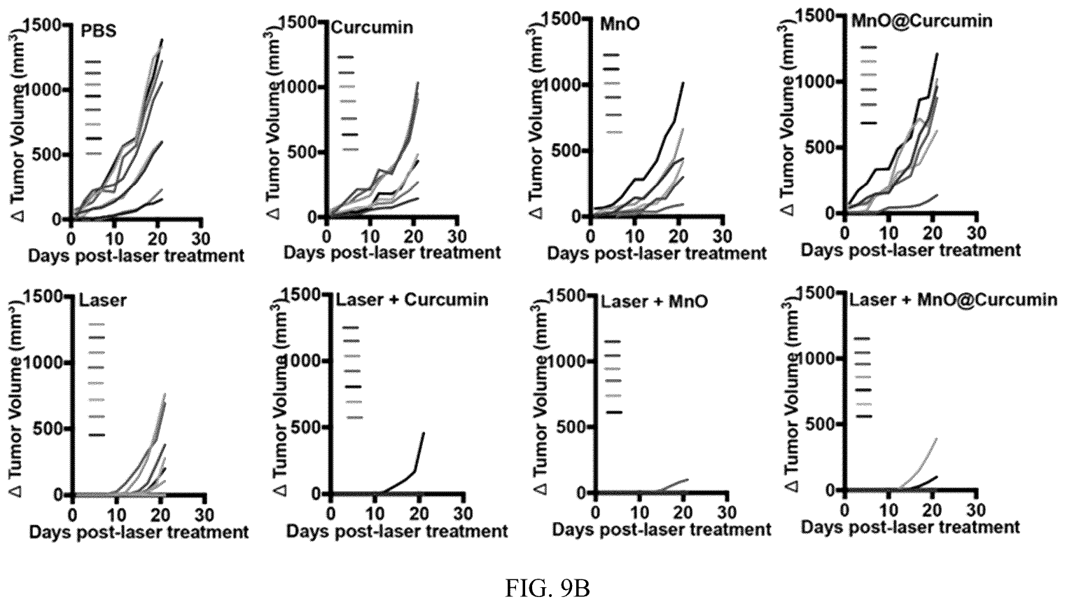

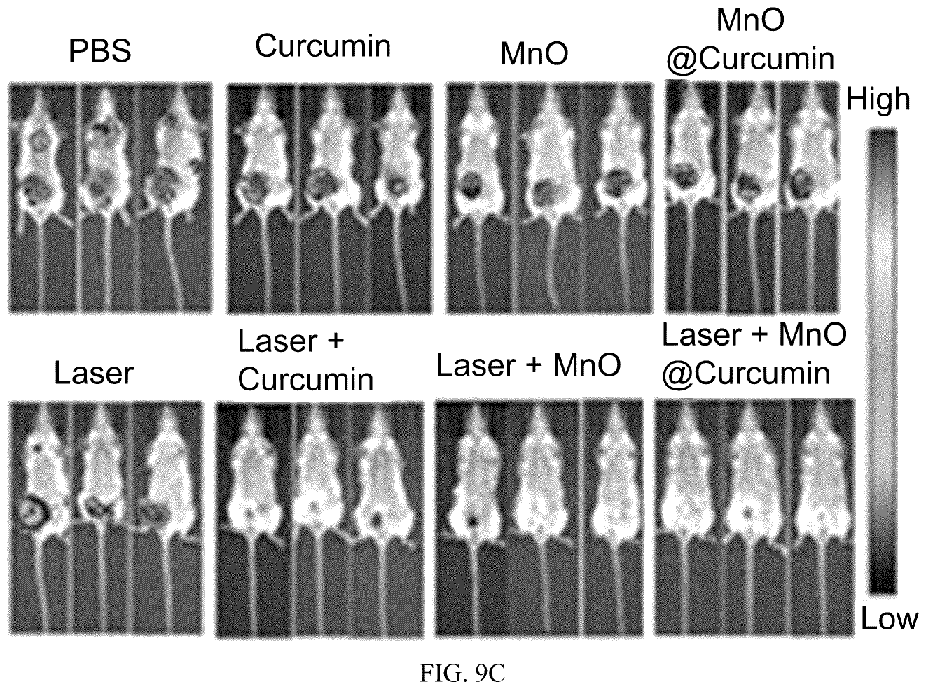

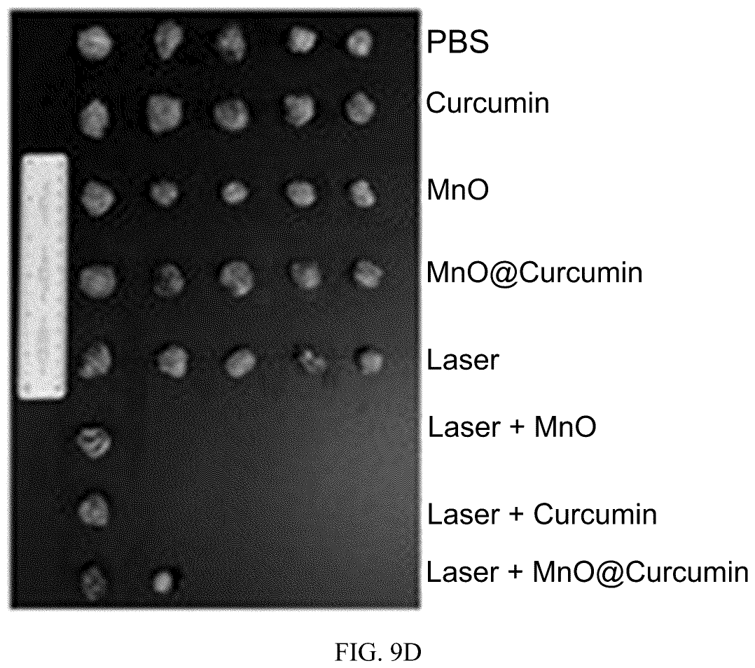

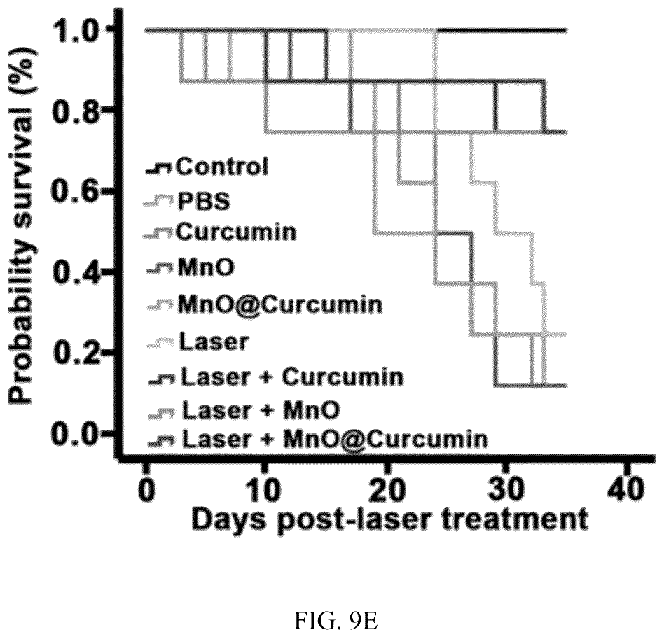

The ability of the MnO and MnO@Cucumin nanoparticles to mitigate the PTT-induced inflammatory response was examined on 4T1 tumor-bearing mice. When the tumor volume reached nearly 100 mm³, 4T1 tumor-bearing mice were randomly divided into 8 groups (n=8 per groups) and treated with one of the following: PBS, MnO, curcumin, MnO@Curcumin, laser, laser+curcumin, laser+MnO, and laser+MnO@Curcumin. The 4T1 tumor-bearing mice received photothermal therapy first, and then were further treated with different formulations by intravenous injection three times per week. As illustrated in FIGS. 9A and 9B, only the curcumin, MnO, and MnO@Curcumin groups did not show obvious effects on the body weight of the mice and the size of the tumors. Though the laser group exhibited increased body weight, tumor recurrence was observed after 15 days post PTT therapy. In contrast, mice in the laser+curcumin, laser+MnO, and laser+MnO@Curcumin groups displayed increased body weight and inhibited tumor recurrence and metastasis, thus confirming that curcumin, MnO nanoparticles, and MnO@Curcumin nanoparticles can be used to inhibit inflammatory responses.

IVIS® images of mice treated with different formulations are shown in FIG. 9C, which further demonstrates the therapeutic effects of MnO and MnO@Curcumin nanoparticles with regard to PTT-induced inflammation. Additionally, five out of eight tumors in the MnO@Curcumin nanoparticle group completely disappeared after treatments, as shown in FIG. 9D, proving that a combination of cfDNA scavengers and anti-inflammation agents has potential for inhibiting tumor recurrence.

### Methods of Inhibiting Viral Infection and Viral-Induced Inflammation

The present invention is directed to methods for using the MnO nanoparticles to (1) reduce or inhibit viral infection of cells, (2) reduce or inhibit viral infection of a subject, and (3) reduce inflammation in a subject.

In a first aspect, the present invention provides methods of reducing or inhibiting viral infection of cells. The methods comprise contacting the cells with a MnO nanoparticle. “Contacting” as used herein, e.g., as in “contacting a sample or contacting cells” refers to contacting a sample or cells directly or indirectly in vitro, ex vivo, or in vivo (i.e. within a subject as defined herein). Contacting a sample or cells may include addition of a compound to a sample, addition to cells, addition to a tissue, addition to a site on a subject, such as coating or adding to the nasal or mucosal passages of a subject or administration to a subject. Contacting encompasses administration to a solution, cell, tissue, mammal, subject, patient, or human. Further, contacting a cell includes adding an agent to a cell culture.

A “viral infection” is an infection caused by the presence of a virus in cells or in a subject. Viral infection can be assessed using a variety of known methods. For example, viral infection can be assessed using an immunoassay to detect viral protein (i.e., using a virus-specific antibody) or using PCR or nucleic acid sequencing to detect viral genomic material.

The methods of the present invention may reduce or inhibit viral infection of cells by at least 50%, at least 60%, at least 70%, at least 80%, at least 90%, at least 95%, at least 98%, or more as compared to untreated control cells, i.e., comparable cells that were not contacted with the MnO nanoparticles. The Examples demonstrate an 80% reduction in plaque formation after cells are contacted with the MnO nanoparticles. The reduction in or inhibition of viral infection may be a reduction in the risk of infection or spread of viral infection when the method is applied prophylactically as compared to control untreated cells, i.e., to a cell population prior to infection to reduce the infection and/or spread of the virus from cell to cell. The reduction in viral infection may result in a reduction in viral entry, viral replication, viral cell-to-cell spread as compared to cells not treated with the compositions provided herein. For example, as shown in the Examples the plaque number and/or size may be reduced.

Most viruses enter host cells by binding to a cell surface receptor and undergoing endocytosis. In the Examples, the inventors demonstrate that their MnO nanoparticles can prevent SARS-CoV-2 viral entry, replication, and cell death. Without being bound by theory, the inventors believe that the nanoparticles may produce this effect by inhibiting the interaction between the viral spike protein and the ACE-2 receptor on the host cell by surrounding the spike protein with charged carboxyl and amine groups and creating a steric barrier.

A “cell” is the basic unit from which all living things are composed. Every cell consists of cytoplasm (i.e., gelatinous liquid that fills the inside of the cell) enclosed within a membrane.

In the methods of the present invention, the cells may be contacted with the MnO nanoparticle directly or indirectly. In some embodiments, the cells are contacted with the MnO nanoparticle in vitro or ex vivo. For example, the cells may be contacted with the MnO nanoparticle in tissue culture (e.g., in a tissue culture plate or flask) by adding the MnO nanoparticle to the culture medium. In other embodiments, the cells are contacted with the MnO nanoparticle in vivo, i.e., in a subject. In these embodiments, the contacting step may be performed by administering the MnO nanoparticle to the subject.

In a second aspect, the present invention provides methods of reducing or inhibiting a viral infection of a subject. The methods comprise administering a MnO nanoparticle to the subject.

Reduction or inhibition of a viral infection in a subject can be detected as a lack of infection or as a lower viral load, reduced severity of symptoms, or faster recovery as compared to an untreated control subject, i.e., a comparable subject that did not receive a dose of the MnO nanoparticles. The methods of the present invention may reduce or inhibit viral infection of a subject by at least 50%, at least 60%, at least 70%, at least 80%, at least 90%, at least 95%, at least 98%, or more as compared to an untreated control subject. The reduction in or inhibition of viral infection may be a reduction in the risk of infection or of morbidity associated with viral infection when the method is applied prophylactically as compared to control untreated subjects, i.e., to an uninfected individual at high risk of infection such as a nurse or doctor working with or exposed to COVID infected patients. The reduction in viral infection may result in a reduction in severity or length of disease/morbidity in an infected subject as compared to subjects not contacted or administered the compositions provided herein. For example, the infected individual may use the methods described herein to shorten the course of disease for the infected individual and thus administration may be after diagnosis with the infection in the subject.

The viral infection may be caused by any kind of virus. In some embodiments the virus is capable of infecting the human respiratory tract. Suitable examples of such viruses include influenza, coronaviruses, rhinoviruses, respiratory syncytial virus, and the like. Examples of coronaviruses include human coronavirus 229E (HCoV-229E), human coronavirus NL63 (HCoV-NL63), human coronavirus OC43 (HCoV-OC43), human coronavirus HKU1 (HCoV-HKU1), Middle East respiratory syndrome coronavirus (MERS-CoV), severe acute respiratory syndrome coronavirus (SARS-CoV), and severe acute respiratory syndrome coronavirus 2 (SARS-CoV-2). In preferred embodiments, the virus is SARS-CoV-2.

The MnO nanoparticle may be administered to the subject either prophylactically or therapeutically. For example, in some embodiments, the subject is simply at high risk of exposure to a virus. For example, the subject may live or work in a setting where COVID-19 is common, such as a nursing home, hospital, or meat packing facility. In some embodiments, the subject is believed to have been exposed to a virus. For example, the subject may have had close contact with someone who is known to have been infected with the virus. In some embodiments, the subject is known to have been infected by a virus. In these embodiments, the subject may show symptoms (e.g., fever, chills, cough) or be asymptomatic.

In a third aspect, the present invention provides methods of reducing inflammation in a subject. The methods comprise administering a MnO nanoparticle to the subject.

“Inflammation” is the immune system's natural response to harmful stimuli such as pathogens, damaged cells, toxic compounds, or irradiation. In some embodiments, the inflammation treated by the method is associated with a viral infection, an autoimmune disease, a cancer, trauma, or sepsis. In preferred embodiments, the inflammation is due to COVID-19.

A reduction in inflammation can be assessed by measuring the expression of inflammatory markers. A reduction in inflammation can also be detected as a reduction in symptoms of inflammation such as swelling, redness, or pain, or as a reduction in a symptom of a specific inflammatory disease. In COVID a reduction in inflammation may be measured as maintenance of oxygen saturation of the blood of the subject and maintenance of normal lung function. The MnO nanoparticles of the present invention reduce inflammation by blocking DAMP/PAMP-mediated activation of TLR signaling. Thus, in some embodiments, a reduction in inflammation is assessed by measuring the expression or activation of a downstream effector of a TLR signaling pathway. The methods of the present invention may reduce or inhibit inflammation in a subject by at least 50%, at least 60%, at least 70%, at least 80%, at least 90%, at least 95%, at least 98%, or more as compared to a similar measured inflammation response in an untreated control subject. The reduction of inflammation may be a reduction in the symptoms of infection, the morbidity associated with viral infection, the duration of morbidity or infection in the subject, or any other measure of inflammation in the subject when the method is applied prophylactically as compared to control untreated subjects, i.e., when the composition is administered to an uninfected individual, such as an individual at high risk of infection such as a nurse or doctor working with or exposed to COVID infected patients. The reduction in viral induced inflammation may result in a reduction in severity or length of disease/morbidity in an infected subject as compared to subjects not contacted or administered the compositions provided herein therapeutically (i.e., after diagnosis). For example, the infected individual may use the methods described herein to shorten the course of disease for the infected individual and thus administration may be after diagnosis with the infection in the subject.

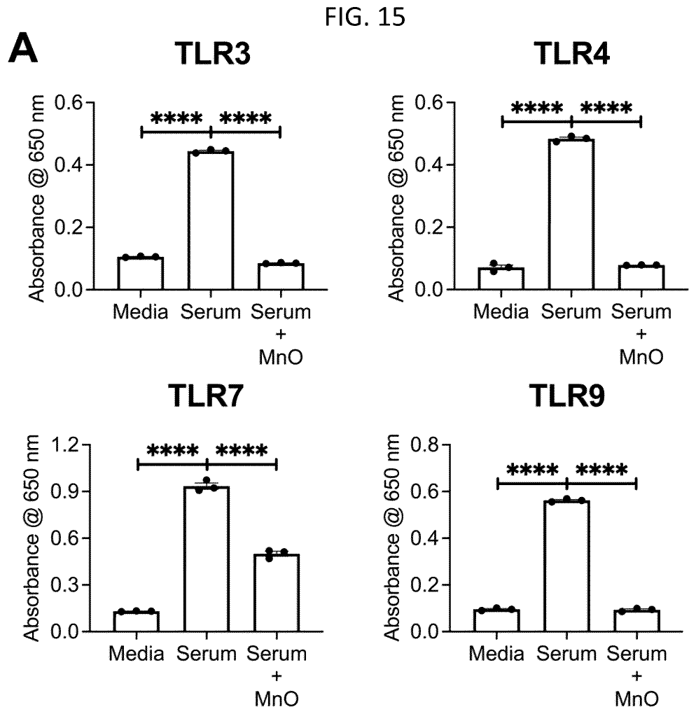

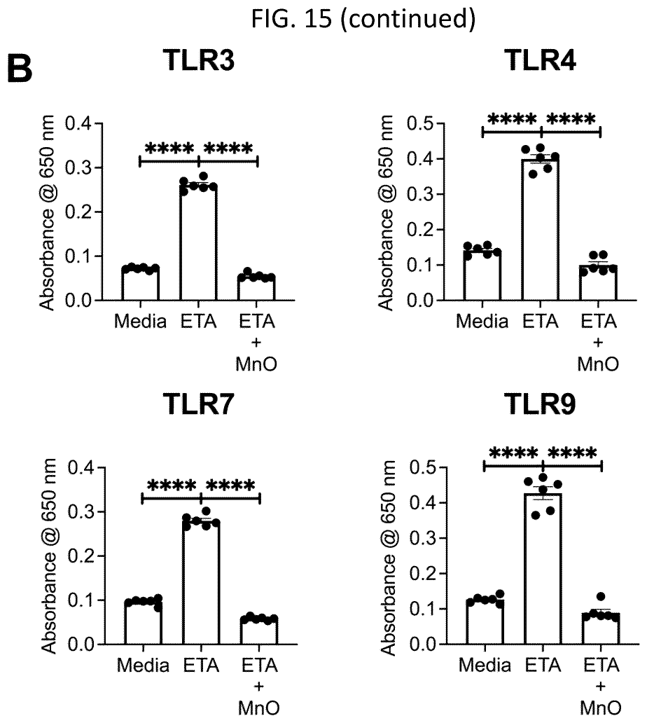

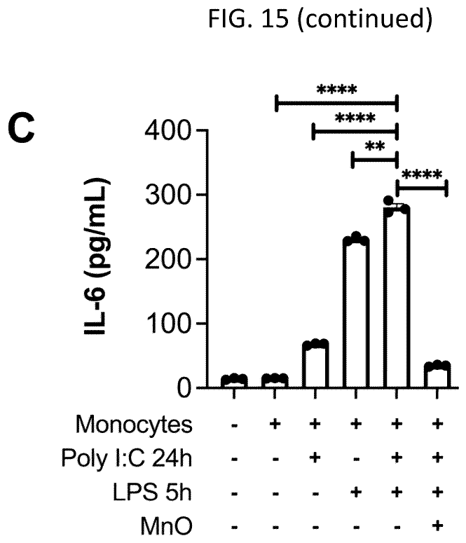

The methods of the present invention can be used to reduce inflammation that is due to activation of Toll-like receptor (TLR)-signaling by nucleic acid-containing DAMPs/PAMPs. In the Examples, the inventors demonstrate that MnO nanoparticle addition significantly reduced stimulation of TLR3, TLR4, TLR7, and TLR9 by the DAMPs/PAMPs present in COVID-19 ICU patient serum and ETA (FIG. 15A-B). Thus, in some embodiments, the methods prevent or reduce activation of one or more nucleic acid-sensing TLRs in the subject. In preferred embodiments, the methods prevent activation of TLR3, TLR4, TLR7, and/or TLR9.

When immune cells, such as monocytes, are repeatedly exposed to TLR agonists, they become “tolerant” to further stimulation and cannot mount an effective response. “TLR tolerance” can be described as a state of altered responsiveness of cells to the repeated or chronic activation of TLRs. In the Examples, the inventors demonstrate that treatment with the MnO nanoparticles significantly reduces IL-6 production by monocytes in response to sequential TLR stimulation (FIG. 15C). Higher levels of secreted IL-6 are indicative of a TLR tolerant phenotype. Thus, this work demonstrates that the MnO nanoparticles limit induction of a TLR tolerance phenotype. Therefore, in some embodiments, the methods prevent the development of TLR tolerant monocytes in the subject. TLR tolerance can be detected in monocytes as reduced production of IL-10, IL-1β, and IL-12 or increased levels of IL-6 production.

The inventors also determined that CD16+ monocytes from deceased COVID-19 patients exhibit a TLR-tolerant phenotype. While monocytes from recovered patients produce a robust anti-viral response to TLR activation, monocytes from deceased patients mount a deficient anti-viral response that amplifies the effects of the cytokine storm. Thus, in some embodiments, the methods prevent the development of a cytokine storm. A “cytokine storm” is a physiological reaction in which the innate immune system causes an uncontrolled and excessive release of pro-inflammatory signaling molecules called cytokines. Normally, cytokines are part of the body's immune response to infection, but their sudden release in large quantities can cause multisystem organ failure and death.

Any of the methods described herein may be performed on a subject. The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods are performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal/subject. Methods of administration are well known in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent. In the Examples, the inventors tested the ability of the MnO nanoparticles to reduce infection and inflammation caused by SARS-CoV-2, which primarily infects tissue lining the airways. Thus, in some embodiments, the MnO nanoparticles are administered orally, nasally, or topically.

In preferred embodiments, the MnO nanoparticles are administered in a therapeutically effective amount, i.e., an amount sufficient to effect beneficial or desirable biological or clinical results. That result can be reducing, alleviating, inhibiting, or preventing inflammation or one or more symptoms of a disease or condition (e.g., a viral infection). Methods for determining an effective means of administration and dosage are well known to those of skill in the art and will vary with the formulation used for therapy, the purpose of the therapy, and the subject being treated. Single or multiple administrations can be carried out with the dose level and pattern being selected by the treating physician.

As used herein, the term “viral infection” refers to those conditions characterized by the presence of a virus in the body (of a subject). The subject may show symptoms (e.g., fever, chills, stuffy/runny nose, etc.) or be asymptomatic. The methods and formulations provided herein are also useful in the prevention of a viral infection and may be used as a prophylactic for a subject at risk of developing a viral infection (e.g., a medical professional). The viral infection may be caused by any kind of virus. In some embodiments, the virus is capable of infecting a human subject. In some embodiments the virus is capable of infecting the mucosal/nasal/lungs/respiratory tract. Suitable examples include influenza, coronaviruses, rhinoviruses, respiratory syncytial virus, and the like. In some embodiments, the viral agent is caused by a coronavirus. In other embodiments, the coronavirus is selected from the group consisting of 229E (alpha coronavirus), NL63 (alpha coronavirus), OC43 (beta coronavirus), HKU1 (beta coronavirus), MERS-CoV (beta coronavirus), SARS-CoV (beta coronavirus), SARS-CoV-2, and combinations thereof. In one embodiment, the coronavirus comprises SARS-CoV-2.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

Millions of COVID-19 patients have succumbed to respiratory and systemic inflammation. Hyperstimulation of toll-like receptor (TLR) signaling is a key driver of immunopathology following infection by viruses. As is described in the following Example, the inventors found that severely ill COVID-19 patients in the intensive care unit (ICU) display hallmarks of such hyperstimulation with abundant agonists of nucleic acid-sensing TLRs present in their blood and lungs. The inventors show that these nucleic acid-containing damage and pathogen associated molecular patterns (DAMPs/PAMPs) can be depleted using nucleic acid-binding microfibers, and that this depletion limits the patient samples' ability to hyperactivate such innate immune receptors. They used single-cell RNA sequencing to reveal that CD16⁺ monocytes from deceased but not recovered ICU patients exhibit a TLR-tolerant phenotype and a deficient anti-viral response after ex vivo TLR stimulation, and they used plasma proteomics to confirm such myeloid hyperactivation and reveal DAMP/PAMP carrier consumption in deceased patients. They found that treatment of these COVID-19 patient samples with MnO nanoparticles effectively neutralizes TLR activation by the abundant nucleic acid-containing DAMPs/PAMPs present in their lungs and blood. Finally, they determined that MnO nanoscavenger treatment limits the ability of DAMPs/PAMPs to induce TLR tolerance in monocytes. Thus, treatment with microfiber- or nanoparticle-based DAMP/PAMP scavengers may prove useful for limiting SARS-CoV-2 induced hyperinflammation, preventing monocytic TLR tolerance, and improving outcomes in severely ill COVID-19 patients.

Several groups have studied hyperactive innate immune responses driven by DAMPs/PAMPs released from dead and dying cells in a variety of disease types [[1], [2], [3]]. DAMPs and PAMPs are detected by pattern recognition receptors, such as Toll-like receptors (TLRs). TLRs are key innate immune sensors, several of which recognize nucleic acid-containing DAMPs/PAMPs to initiate signaling cascades and downstream immune responses [4,5]. TLR3 and TLR7/8 sense RNA, whereas TLR9 senses DNA and TLR4 senses DNA-protein complexes. Together, these nucleic acid-sensing TLRs are important mediators of the inflammatory response in cancer, autoimmune diseases, and infections [[5], [6], [7], [8]]. However, excessive activation of TLRs and their downstream signaling pathways via NFκB may contribute to the development and progression of lung injury and acute respiratory distress syndrome (ARDS) due to increased vascular permeability and lymphocyte and neutrophil activation [9,10]. We and others have previously shown that both the soluble and non-soluble fiber formulations of certain nucleic acid-binding micro- and nano-materials can neutralize such DAMPs/PAMPs and prevent their activation of TLRs and downstream NFκB in models of lupus, rheumatoid arthritis, cancer, trauma, sepsis and influenza infection.

Infection by SARS-CoV-2 causes a broad spectrum of disease, from mild symptoms of upper respiratory infection to ARDS and respiratory failure requiring mechanical ventilation or extracorporeal membrane oxygenation [[23], [24], [25]]. Patients at the severe end of the disease spectrum, such as those in the intensive care unit (ICU), can quickly deteriorate and are at higher risk of mortality [23,25,26]. Underpinning these poor outcomes is a hyperinflammatory response in severely ill patients concurrent with a viral infection, which is associated with elevated levels of D-dimer, IL-6, lymphopenia, and neutrophilia among other laboratory abnormalities [24,26]. We reasoned that the TLR hyperstimulation we have studied in other disease types may be relevant to the hyperinflammatory response in COVID-19. Therefore, in this study, we set out to characterize TLR-mediated inflammation in ICU patients with COVID-19 and test the ability of nucleic acid-binding materials to mitigate the negative downstream consequences of nucleic acid-containing DAMPs/PAMPs in this patient population.

TLR activation by DAMPs and PAMPs results in altered expression of cell surface receptors, cytokines, and chemokines. SARS-CoV-2 infection in ICU patients is associated with marked neutrophilia, with neutrophils also being a key source of DAMPs in this population of patients [27]. Some reports identified an increase in monocyte abundance associated with severe infection [28,29]. Monocytes are key sensors of and responders to TLR-mediated inflammation, especially following viral infection. Classical monocytes (i.e., CD16⁻CD14⁺ monocytes) typically represent the majority of circulating monocytes under healthy conditions (80-95% of total monocytes); intermediate (CD16⁺CD14⁺) and non-classical monocytes (CD16⁺CD14^{lo/-}) comprise the remaining 20-5% [30,31]. CD16⁺ monocytes respond to TLR7/8-mediated inflammation, and CD16⁻ monocytes respond to TLR4-mediated inflammation [31].

Under normal circumstances, TLR activation results in downstream signaling and changes in gene transcription that allow monocytes to respond to infection. However, severe infection and a hyperinflammatory environment can lead to hyperstimulation of TLRs and subsequently to aberrant TLR downstream signaling. This persistent and aberrant downstream TLR signaling in the setting of hyperinflammation is referred to as “TLR tolerance”. TLR tolerance occurs when cells are repeatedly exposed to DAMPs and PAMPs [15, [32], [33], [34], [35], [36]]. Under normal circumstances, the TLR signaling cascade activates NFκB and leads to a robust immune response. However, when immune cells, such as monocytes, are repeatedly exposed to TLR agonists, they become tolerant to further stimulation and cannot mount an effective response [[15], [32], [33], [34], [35], [36]]. Previous studies have shown that monocytes isolated from patients with sepsis have reduced production of IL-10, IL-1β, and IL-12 in response to LPS treatment, regardless of patient survival [37]. However, when monocytes were isolated and activated with LPS later in the course of sepsis, monocytes from survivors regained the ability to produce these cytokines in response to LPS treatment, whereas monocytes from deceased patients did not recover. Moreover, this cytokine dysfunction could not be rescued with in vitro treatment of IFN-γ. This phenotype predisposes these patients to subsequent bacterial infections given the importance of IL-10, IL-1β, and IL-12 in the resolution of inflammation [[37], [38], [39], [40]]. We, therefore, examined if monocytic TLR tolerance occurs in COVID-19 patients in response to nucleic acid-containing DAMPs/PAMPs and whether nucleic acid-binding nanomaterial-based therapies could mitigate TLR hyperactivation by patient samples and limit DAMP/PAMP-induced tolerance of monocytes.

To this end, we conducted longitudinal immune and DAMP/PAMP profiling of patients with COVID-19 requiring ICU-level care. We examined immune subsets in blood from critically ill COVID-19 patients using flow cytometry, used reporter cell assays to measure TLR activation potential, and used single-cell RNA-sequencing and plasma proteomic profiling to assess differences between patients that survived versus those that did not. We observed a striking elevation of nucleic acid-containing DAMPs/PAMPs in patients' lungs and blood and thus investigated the ability of nucleic acid-binding micro- and nanomaterials to neutralize the excessive DAMPs/PAMPs. We found that our DAMP/PAMP scavenger treatment inhibits hyperinflammation and limits TLR tolerance, suggesting that such materials may prove useful for improving outcomes of severely ill COVID-19 patients.

### Results:

Immune Profiling of COVID-19 ICU Patient Blood Reveals that Distinct Monocyte Subsets are Associated with Clinical Outcomes

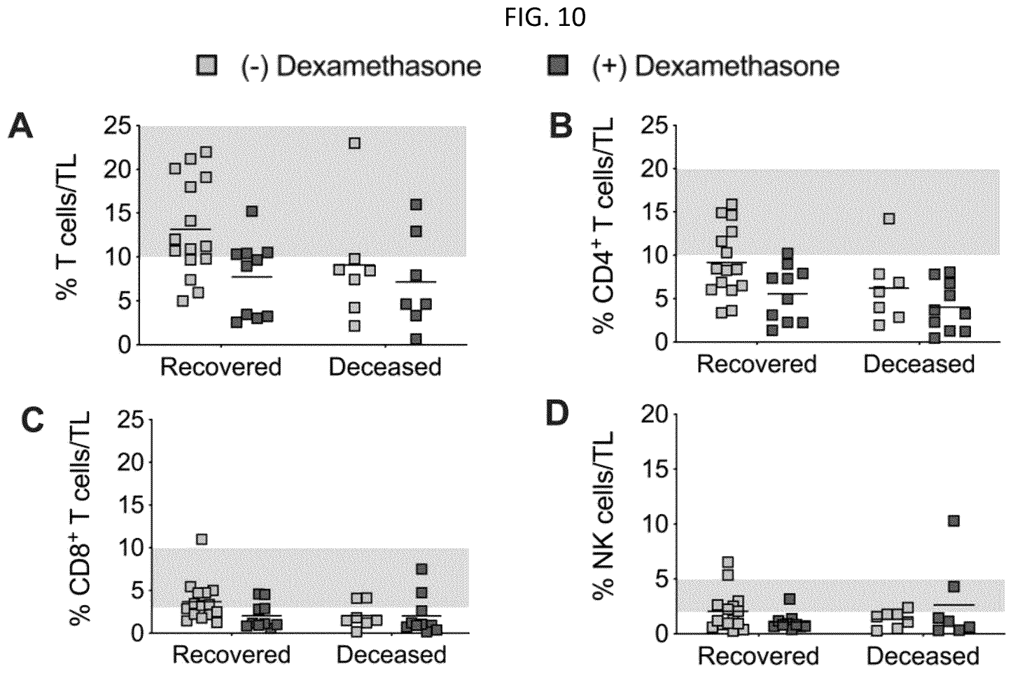

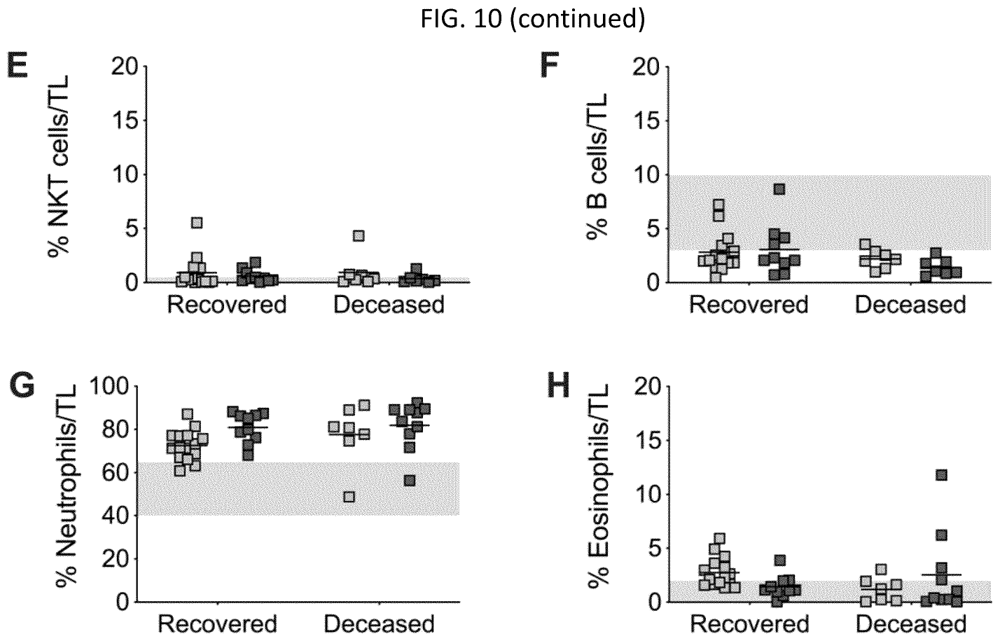

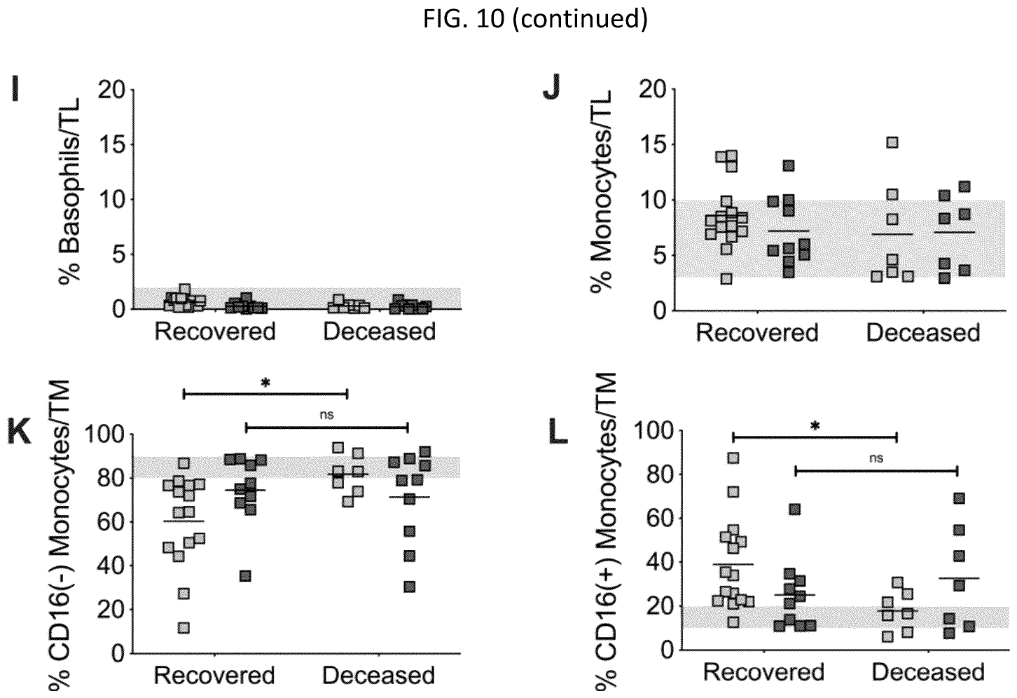

Fresh whole blood was collected from patients at time points indicated and analyzed as described in the Materials and Methods section. Flow cytometry results support the trends observed in previous reports [[23], [24], [41], [42], [43], [44], [45], [46], [47]], including lymphopenia and neutrophilia in most patients, regardless of treatment with dexamethasone or clinical outcome (FIG. 10A-G). When comparing patient groups by dexamethasone treatment and clinical outcomes, no significant differences were observed in the frequency of CD4⁺ and CD8⁺ T cells, NKT cells, NK cells, basophils, or eosinophils (FIG. 10B-I) in the periphery. We observed that a subset of patients had elevated percentages of monocytes while other patients had percentages within the normal range (FIG. 10J). This prompted us to further examine this subset of cells and the canonical subpopulations of monocytes.

Monocyte subsets are categorized by surface expression of CD16 and CD14. Monocytes that express CD16, which are classified as either intermediate monocytes (CD16⁺CD14⁺) or non-classical monocytes (CD16⁺CD14^{lo/-}), were annotated as CD16⁺ monocytes [30,31]. Dexamethasone-naïve ICU patients that recovered from infection were distinguishable from deceased patients based on the abundance of CD16⁺ monocytes (FIG. 10J-L). Patients who were dexamethasone-naïve and recovered from infection had higher levels of CD16⁺ monocytes. Recovery was defined as discharge from the hospital or transition out of the ICU. In contrast, deceased patients had lower levels of CD16⁺ monocytes, and CD16⁻ monocytes were the pre-dominant monocyte population in the periphery of these patients. The association between CD16⁺ monocyte abundance and positive clinical outcome in patients treated with dexamethasone was not statistically significant, exemplifying the potent immune-modulatory effects of dexamethasone. To interrogate the effect of COVID-19 on nascent monocyte function directly, we analyzed samples from the ICU COVID-19 patient cohort that was dexamethasone-naïve. The difference in CD16⁺ cells, which are known to be particularly sensitive to TLR7/8 activation by RNA-based DAMPs/PAMPs, encouraged us to evaluate the levels of such inflammatory nucleic acids in patient samples.

Longitudinal Profiling of Serum and Endotracheal Aspirate from COVID-19 ICU Patients Reveals Elevated Levels of DAMPs and PAMPs that Potently Activate Nucleic Acid-Sensing TLR Signaling

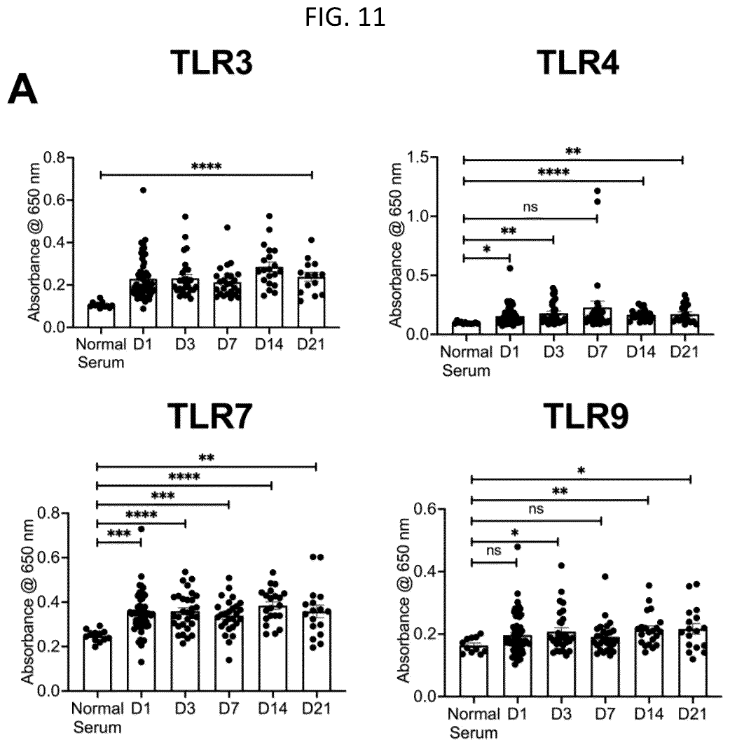

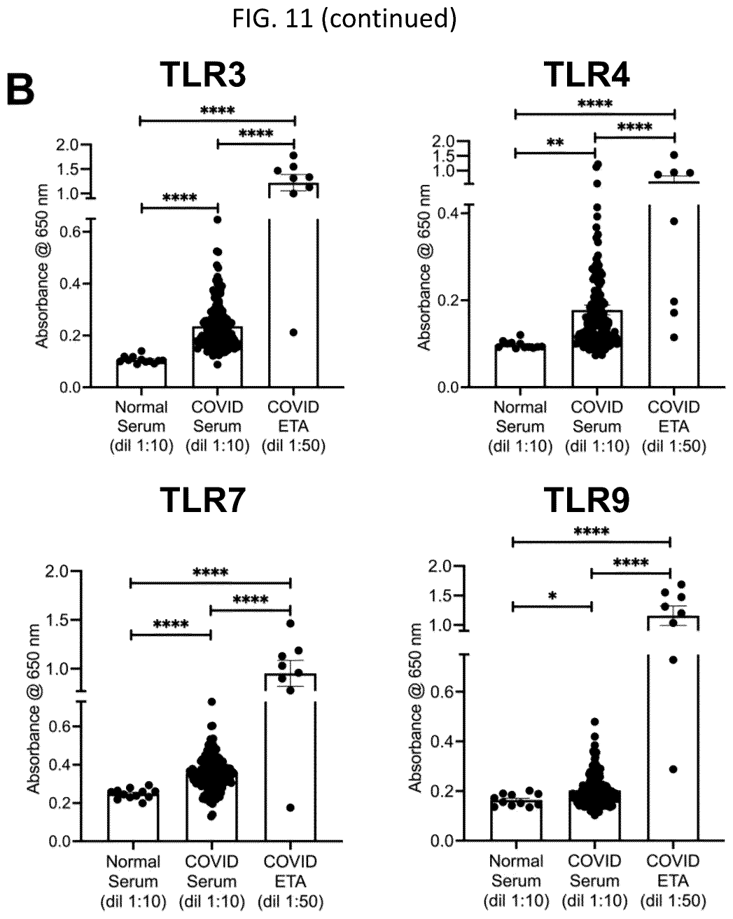

To investigate the associations between CD16⁺ monocytes, TLR activation, and clinical outcome, we analyzed blood, plasma, and endotracheal aspirate (ETA) from ICU COVID-19 patients for TLR agonists. Among the four TLRs tested, TLR3 and TLR7 showed the most consistent activation by serum across all patients and all time points when compared to healthy control (FIG. 11A). Since TLR3 and TLR7 are sensors for double-stranded and single-stranded RNA, respectively, we concluded that serum samples from our COVID-19 ICU cohort have elevated levels of pro-inflammatory RNA molecules, consistent with the fact that SARS-CoV-2 is an RNA virus. TLR4 and TLR9 were also activated by serum from COVID-19 ICU patients, though the level of activation was not as high as found with TLR3 and TLR7, particularly when viewed longitudinally (FIG. 11A). TLR4 and TLR9 are sensors of lipopolysaccharide (LPS) and bacterial DNA, respectively. TLR4 and TLR9 are also activated by endogenous molecules released during cell death such as nucleosomes, HMGB-1, and mitochondrial DNA [48]. We also characterized the TLR profile of ETA from 10 COVID-19 ICU patients. For each TLR tested, ETA produced a 3-4-fold higher activation than serum from 72 COVID-19 patients (FIG. 11B). These data further highlight the respiratory localization of the inflammatory syndrome in patients critically ill with COVID-19. These results indicate that the airway remains a major location of DAMP/PAMP generation and hyperinflammation in these patients characterized by high levels of inflammatory agonists that activate nucleic acid-sensing TLRs, particularly the RNA sensors TLR3 and TLR7/8 which are on CD16⁺ monocytes.

Nucleic Acid-Binding Microfibers can Deplete DAMPs PAMPs from COVID-19 ICU Patient Serum and ETA and Limit their Ability to Stimulate TLRs and Induce Downstream NFκB Activation

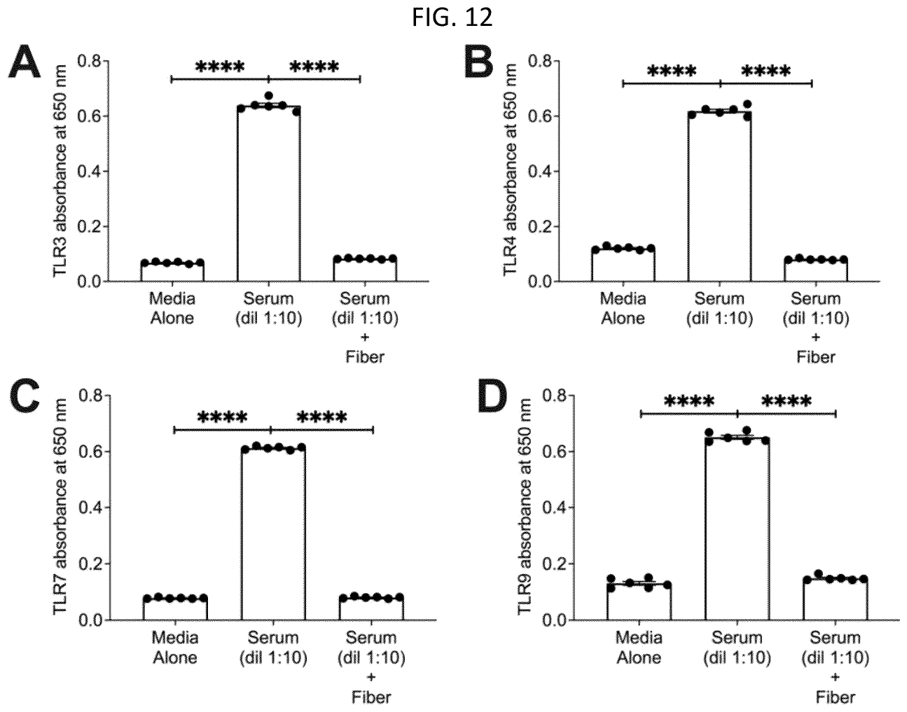

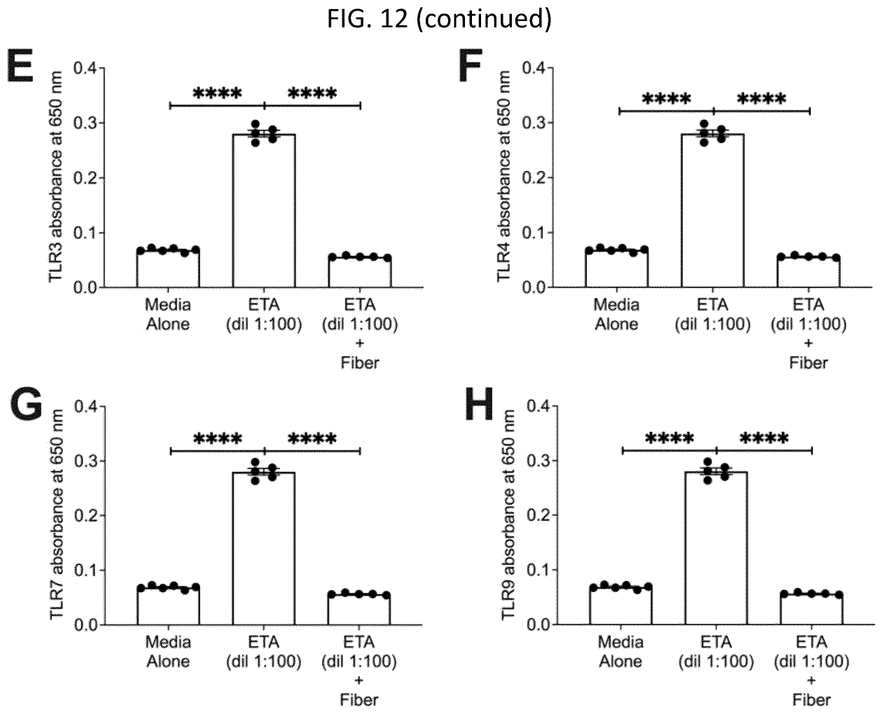

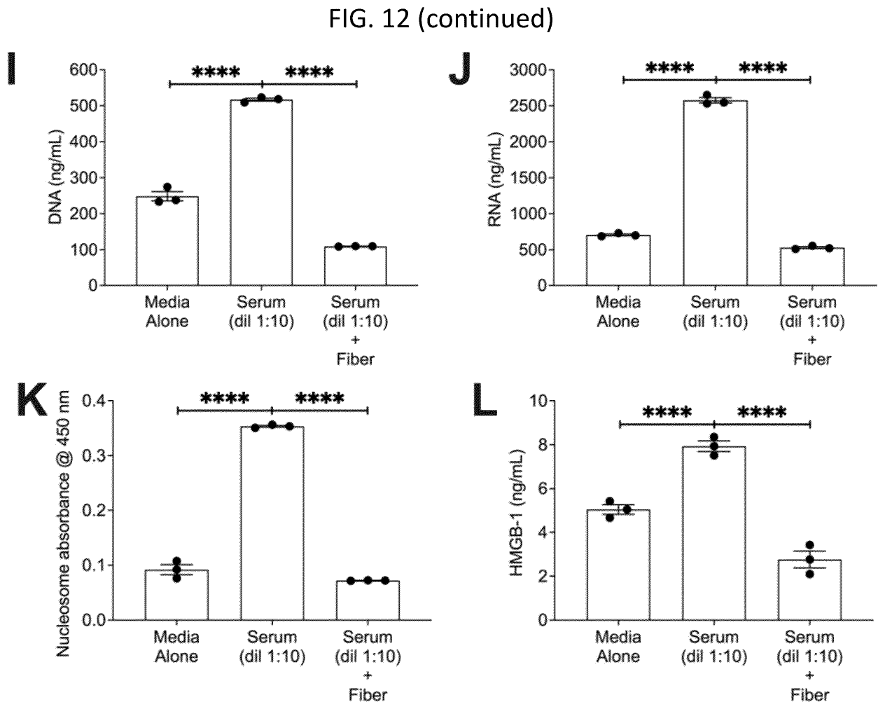

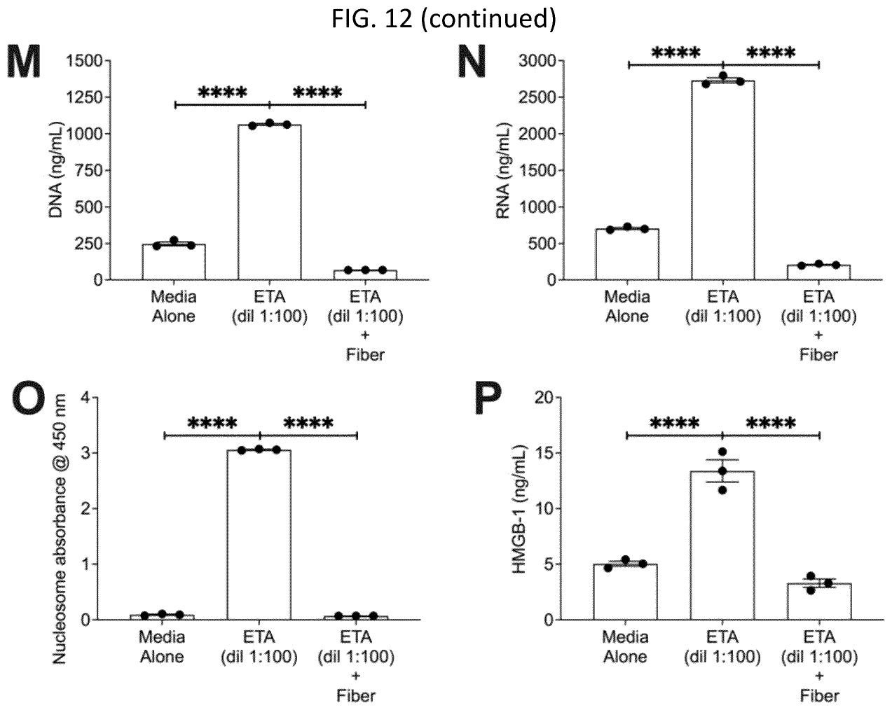

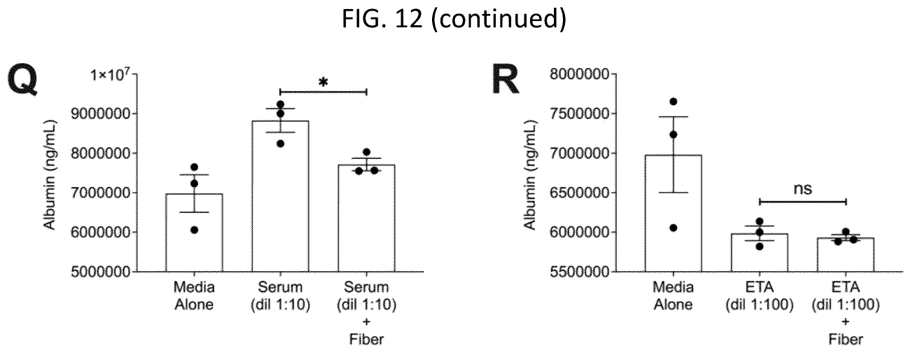

The observation that nucleic acid-containing DAMPs/PAMPs are abundant in COVID-19 ICU patient samples led us to explore if such TLR agonists can be depleted using a nucleic acid-scavenging microfiber mesh. Previously, we observed that such microfibers could effectively remove DAMPs present in the blood of trauma patient samples and limit the ability of such patient samples to stimulate TLRs [15]. Electrospun PSMA/polystyrene microfibers (average diameter 2.5±0.1 μM) were generated and PEI and PAMAM-G3 were conjugated onto the PSMA/polystyrene mesh as previously described [15]. Treatment of patient samples with these nucleic acid-binding microfibers dramatically decreased the activation of TLR3, TLR4, TLR7, and TLR9 by serum and ETA from COVID-19 patients. As shown in FIG. 12A-H, the level of inhibition achieved is essentially complete, as the microfibers reduced levels of TLR activation to those observed with media alone. Thus, treatment with this nucleic acid-binding microfiber mesh neutralized DAMPs and PAMPs and prevented NFκB activation by depleting these TLR agonists from serum and ETA samples collected from blood and lungs of critically ill COVID-19 patients.

We also quantified the ability of the nucleic acid-scavenging fibers to deplete DAMPs/PAMPs from patient samples. We found that treatment with the fibers was able to significantly deplete DNA, RNA, HMGB-1, and nucleosomes from both serum and ETA of COVID-19 patient samples (FIG. 12I-P). (Note: DNA and HMGB-1 are potent endogenous activators of TLR9 [49]. RNA is a canonical activator of TLR3 and TLR7. HMGB-1 and nucleosomes also potently activate TLR4.) We also measured human serum albumin as a non-inflammatory protein control. Though albumin carries a net negative charge, the depletion effect by the nucleic acid-binding fiber is modest in serum and non-existent in ETA (FIG. 12Q-R). These data demonstrate that nucleic acid-containing DAMP/PAMP scavenger fibers can deplete a diverse array of pro-inflammatory molecules present in COVID-19 patient samples while having only a modest effect on a control protein and suggest that such fibers may be useful for depleting DAMPs/PAMPs from COVID-19 patients requiring lifesaving, supportive care such as extracorporeal membrane oxygenation (ECMO).

### Longitudinal Proteomics Reveals Markers of Heightened Myeloid Activation and Consumption of PAMP Carrier Proteins in Deceased Patients

For further validation of the differential expression and functional observations from our RNA-sequencing data, we performed an unbiased proteomics analysis on plasma from four different ICU patients that recovered from COVID-19 and four different patients that did not.

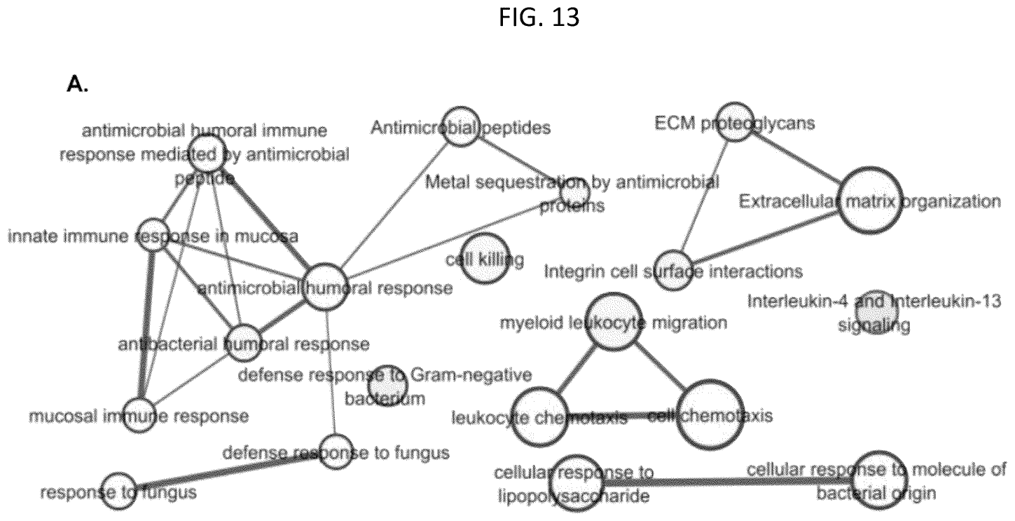

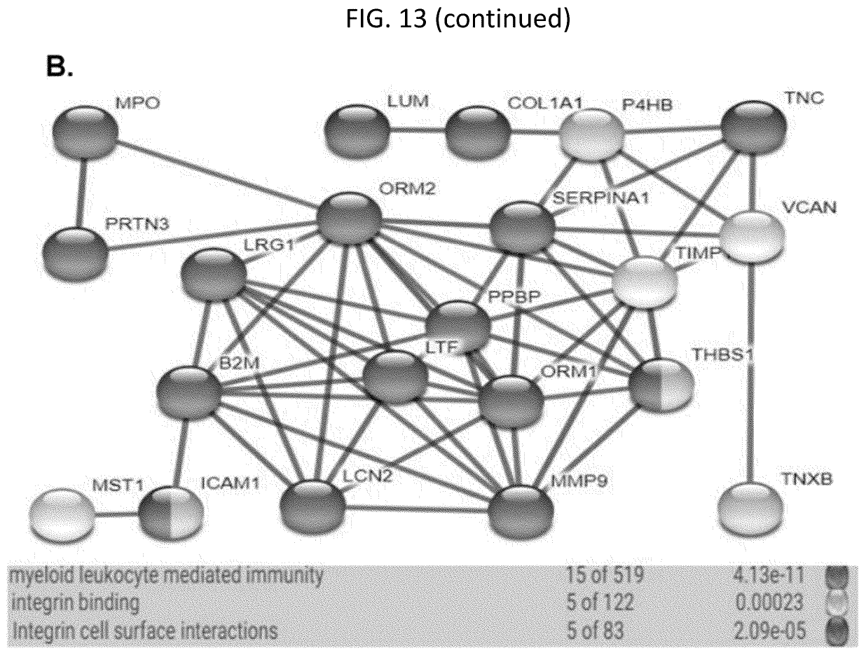

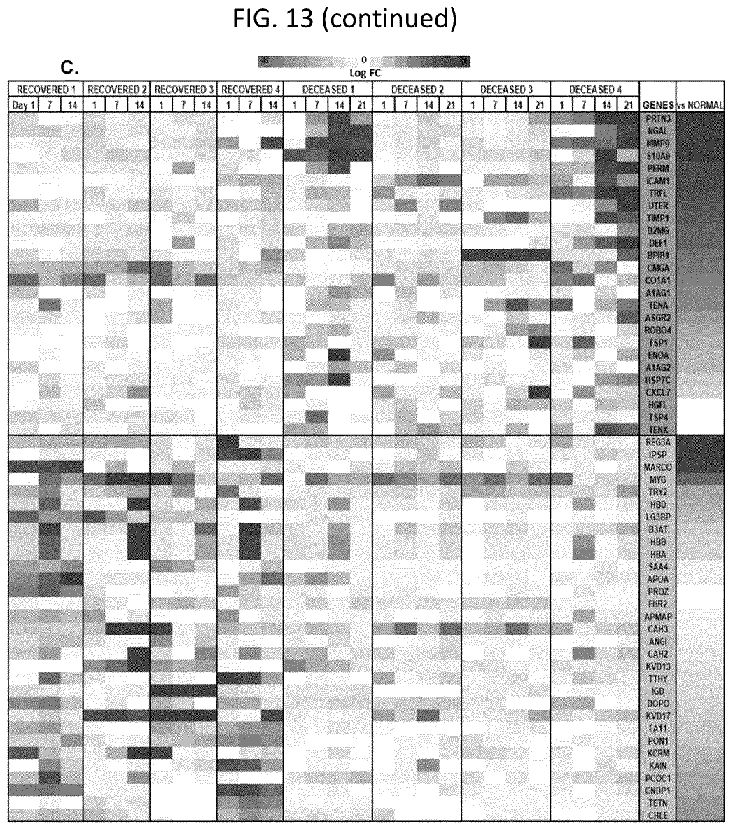

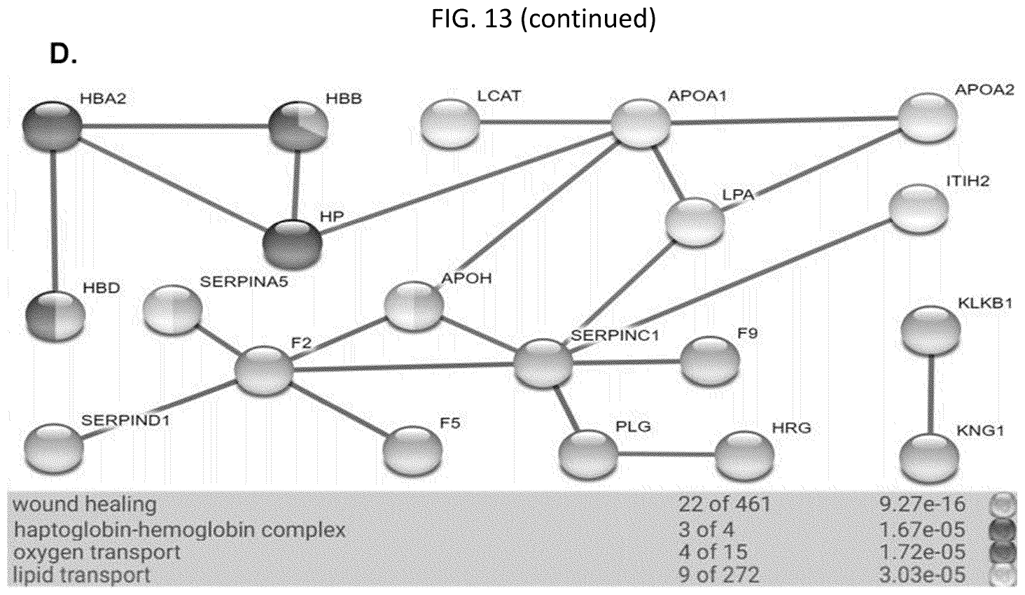

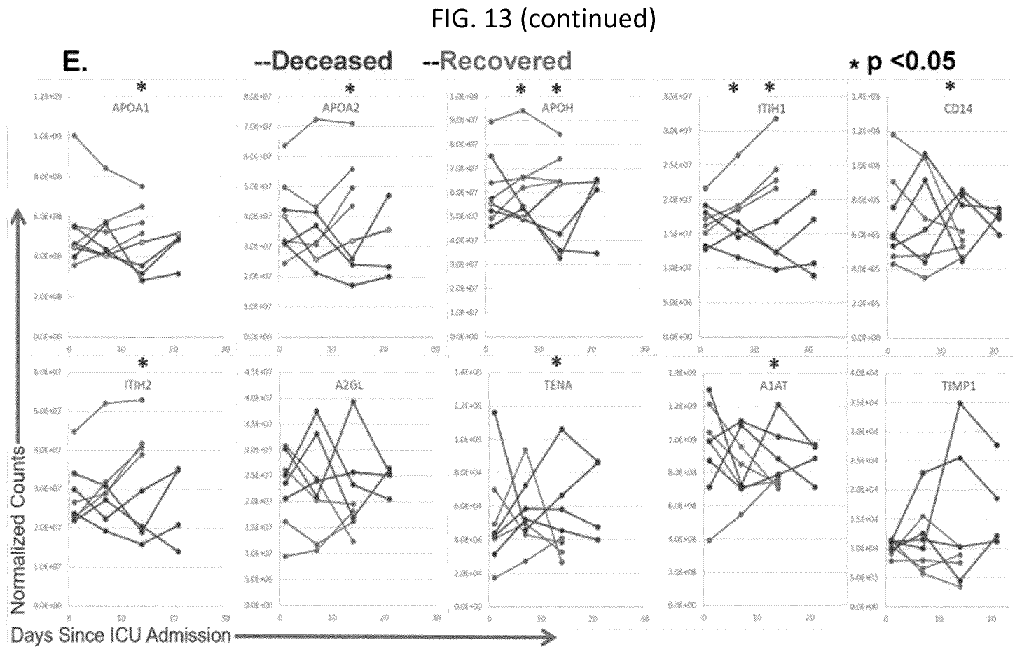

We identified similarities between the circulating proteome from the four deceased patients and the transcriptomic profiles of isolated monocytes from three different deceased patients. Functional pathway analysis performed on the plasma proteome demonstrated an enrichment of biological processes related to myeloid activation, recruitment, and migration in deceased patients relative to survivors (FIG. 13A). Specifically, clustering performed on these proteomic data identified markers of mucosal innate immunity, antibacterial response, and cellular response to lipopolysaccharide in deceased patients. Furthermore, the proteinaceous markers enriched in deceased patients over recovered patients indicated heightened myeloid activity (FIG. 13B).

Next, we compared longitudinal samples from both recovered and deceased patients (FIG. 13C). We observed that deceased patients had higher circulating levels of proteins secreted by activated neutrophils, including NGAL, PRTN3, MMP9, MPO, TRFL, and DEF1 [67]. These proteins are secreted by neutrophils in response to microbial-induced inflammation and are mediated by TLR signaling (FIG. 13C) [[67], [68], [69], [70], [71]]. Additionally, levels of S100A9, an alarmin protein, were also elevated in deceased patients compared to recovered patients (FIG. 13C) [48,72]. S100 proteins are markers of inflammation involved with positive feedback of TLR4 expression [48,72]. These data are consistent with the monocyte RNA sequencing data, which showed that anti-microbial pathways are elevated in cells from deceased patients compared to recovered patients.

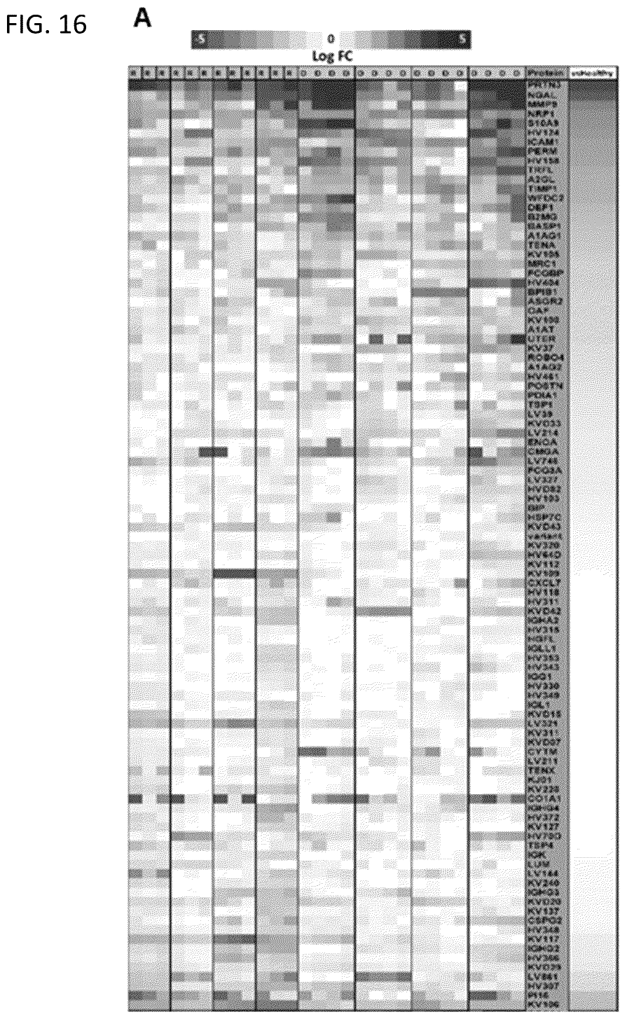

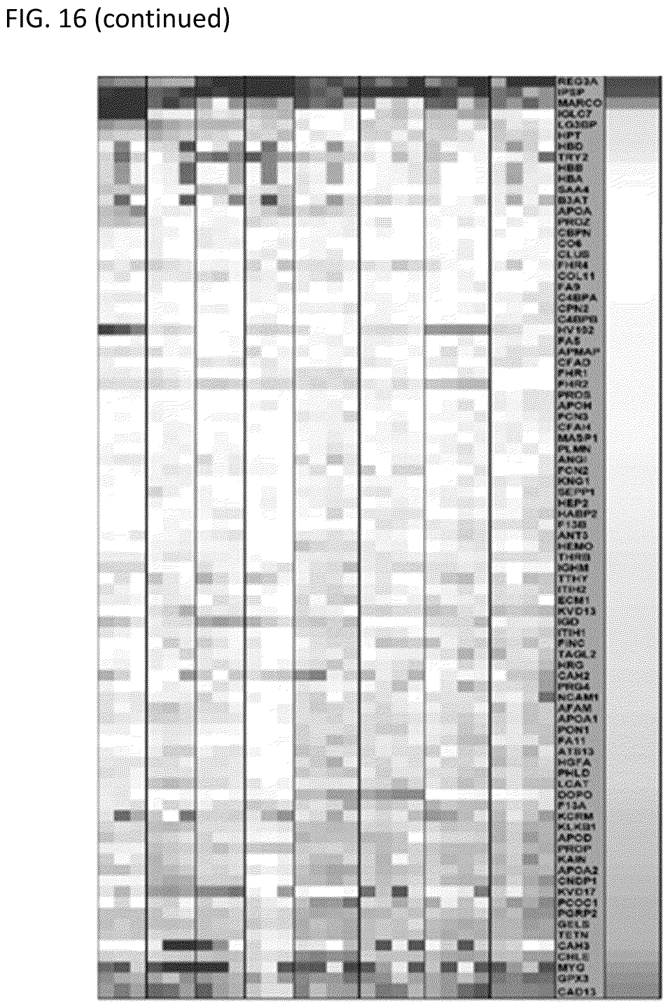

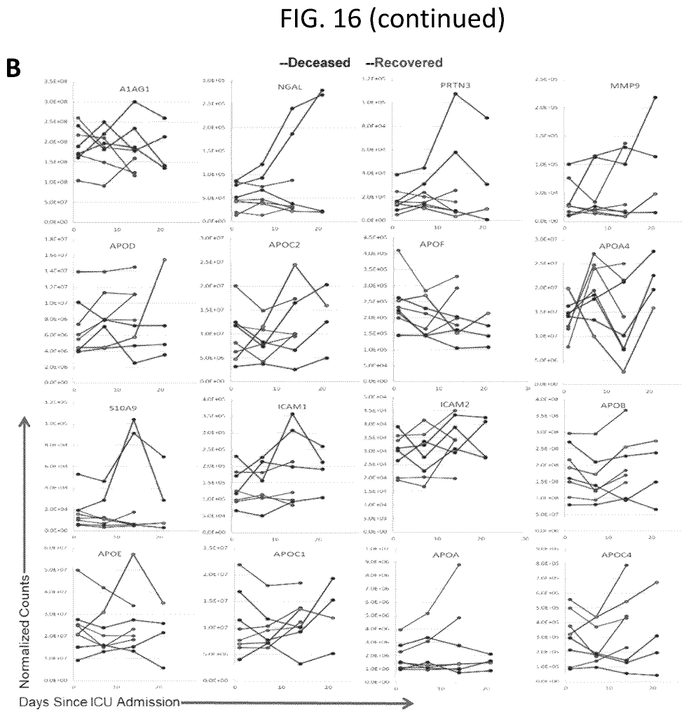

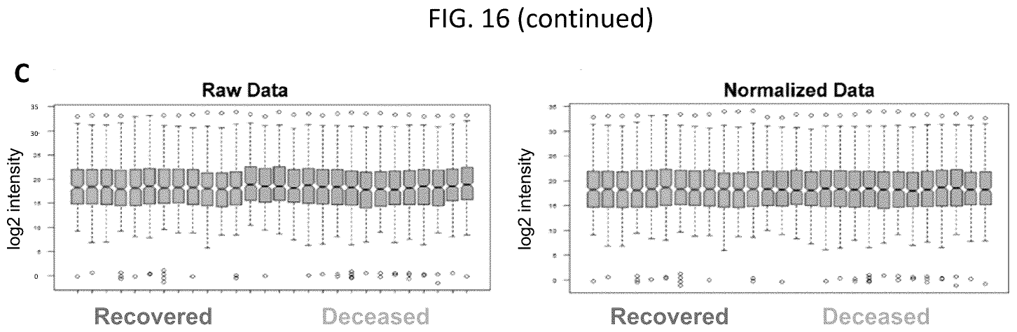

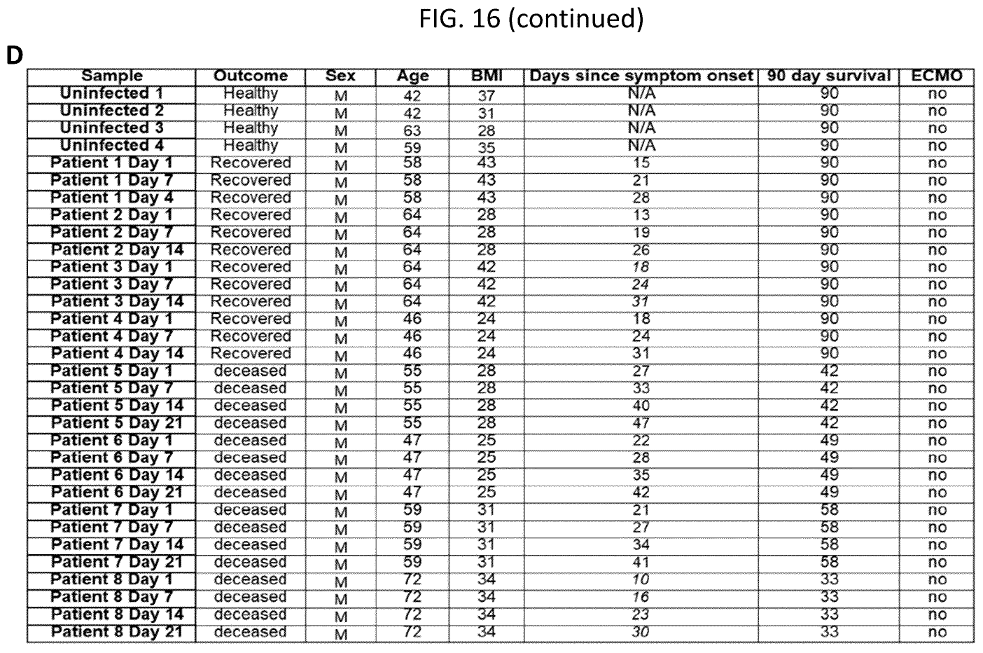

We also identified proteins with higher levels in recovered patients compared to deceased patients. For example, MARCO, a scavenger receptor expressed by macrophages that binds PAMPs in circulation and activates TLR signaling [73,74], is significantly lower in deceased patients relative to recovered patients (FIG. 13C). These findings suggest that PAMP binding and delivery to TLRs is consuming MARCO in deceased patients (FIG. 13C,E). A similar pattern was observed for various apolipoproteins, including APOA1, APOA2, APOH (FIG. 13C,E) as well as APOD and APOC4 (FIG. 16), which have reduced levels in plasma from deceased patients compared to recovered patients (FIG. 13C-E). Apolipoproteins act as carriers for PAMPs to activate TLR signaling and facilitate anti-viral immunity [[75], [76], [77]]. The proteomic evidence that free MARCO and apolipoproteins are decreased, and apparently being consumed, in deceased patient blood is consistent with the transcriptomic findings that monocytes from deceased patients have increased activation of TLRs.

Collectively, the COVID-19 ICU patient DAMP/PAMP TLR reporter cell activation studies, single-cell RNA sequencing, and proteomics analyses indicate that TLR hyperstimulation by nucleic acid-containing DAMPs/PAMPs can lead to excessive monocyte activation and consumption of immune modulators, all of which are associated with poor outcomes for COVID-19 ICU patients. Therefore, we next investigated whether DAMP/PAMP nanoscavengers can limit such TLR hyperactivation and TLR tolerance in monocytes.

### MnO-Based Nanoparticles Mitigate TLR Stimulation by COVID-19 ICU Patient Serum and ETA, and Prevent DAMP PAMP-Mediated Induction of TLR Tolerance in Monocytes

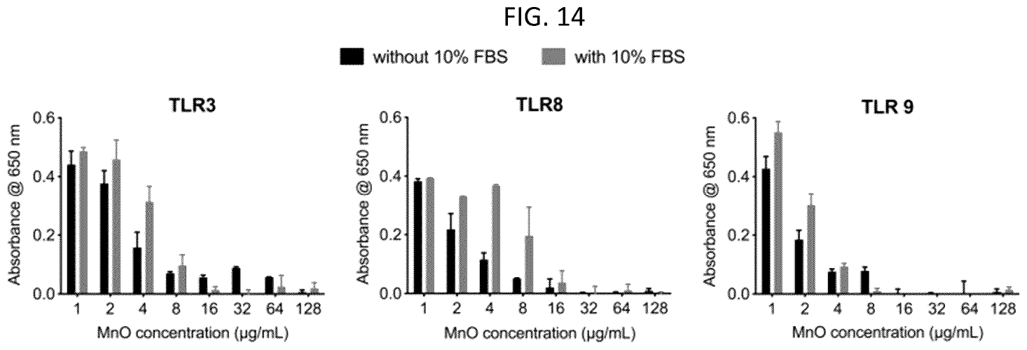

The observation that nucleic acid-binding microfibers can capture and neutralize DAMPs/PAMPs (FIG. 12) led us to evaluate whether a soluble nanoparticle could be identified to counteract DAMPs/PAMPs in COVID-19 patient samples. To this end, we evaluated a new, soluble form of a nucleic acid-binding nanoparticle-based upon manganese oxide (MnO). Given the uncharacterized nature of this MnO nanomaterial, we first assessed its fundamental properties. After synthesis, we determined that the MnO particles generated ranged in size from 30 to 100 nm and have a zeta potential is about −20 mV. We have evaluated the calf thymus DNA binding affinity of the manganese oxide (MnO) nanoparticles in Tris-EDTA (TE) buffer by measuring unbound PicoGreen-labeled DNA. Despite their net negative surface charge, MnO nanoparticles exhibit high DNA binding affinity, which is comparable to the nucleic acid-binding polymer (PAMAM-G3) and have reduced cell toxicity compared to PAMAM-G3 (FIG. 3A). Next, we evaluated the nucleic acid scavenging activity of the MnO nanoparticles using TLR reporter cells. We found that the MnO nanoparticles inhibit CpG-induced activation of HEK-Blue hTLR9 cells, inhibit poly (I:C)-induced activation of HEK-Blue hTLR3 cells, and ORN06/LyoVec (ORN)-induced activation of HEK-Blue hTLR8 cells in a dose-dependent manner, regardless of the presence or absence of FBS (FIG. 14). The MnO-based nanoparticle have several distinct advantages when compared to other soluble nucleic acid-binding nanomaterials we have previously tested. First, MnO nanoparticles maintain a potent ability to bind to inflammatory nucleic acids with a significantly improved toxicity profile when compared to other polymers (FIG. 14). Second, MnO nanoparticles are biodegradable and therefore are not expected to display the same level of toxicity to clearance organs observed with other classes of nucleic acid-binding nanomaterials such as the polyamidoamine and PAMAM families of polymers.

Given these advantages, we wanted to test the utility of such MnO nanoparticles in controlling TLR stimulation by COVID-19 ICU patient samples. As shown in FIG. 15A,B, MnO nanoparticle addition significantly reduced stimulation of TLR 3, 4, 7, and 9 by the DAMPs/PAMPs present in COVID-19 ICU patient serum and ETA. The level of inhibition achieved with the nanoparticle scavengers is essentially 100% and comparable to that achieved using the microfiber DAMP/PAMP scavenger (compare FIG. 12 and FIG. 15). Next, we investigated whether mitigation of TLR stimulation by MnO nanoparticle could prevent induction of TLR tolerance in monocytes ex vivo. To this end, we conducted an experiment in which freshly isolated primary human monocytes were treated with poly I:C (a double-stranded viral RNA PAMP mimic that activates TLR3) for 24 hours, followed by treatment with LPS (a bacterial PAMP mimic that activates TLR4) for 5 hours, after which cell supernatant was collected and the levels of the pro-inflammatory cytokine IL-6 were quantified. Higher levels of secreted IL-6 are indicative of a TLR tolerant phenotype [78]. This experimental setup allowed for the induction of TLR tolerance ex vivo by first exposing monocytes to a viral PAMP mimic (poly I:C) and then to a bacterial PAMP mimic (LPS). This sequential stimulation also recapitulates the inflammatory exposure seen by monocytes in ICU patients with COVID-19, i.e., exposure due to their viral infection followed by any secondary bacterial infection. Using this experimental design, we found that monocytes treated with either LPS or poly I:C alone secreted lower levels of IL-6 when compared to monocytes sequentially treated with poly I:C then LPS. In fact, sequentially treated monocytes produced at least 1.2-fold more IL-6 than monocytes treated with poly I:C or LPS alone. However, the addition MnO nanoparticles was able to completely abrogate IL-6 production in monocytes sequentially treated with poly I:C then LPS. Thus, treatment with the MnO DAMP/PAMP nanoscavenger can significantly reduce IL-6 production by monocytes in response to sequential TLR stimulation and thereby limit induction of a TLR tolerance phenotype (FIG. 15C).

### Inhibition of Agonist-Induced TLRs Activation by MnO Nanoparticles

HEK-Blue hTLR3, hTLR8 and hTLR9 were obtained by co-transfecting the hTLR gene and an optimized secreted embryonic alkaline phosphatase (SEAP) reporter gene into HEK293 cells. HEK-Blue hTLR cells were cultured and maintained in DMEM with 10% FBS and 1% penicillin-streptomycin. To evaluate nanoparticle inhibition of TLR activation, HEK-Blue hTLR cells were seeded in 96-well plate for 1 h then treated with 2 ul of agonist (CPG Bw006 for TLR9, Poly (I:C) for TLR3 or ORN06/Lyo Vec for TLR8, 1 mg/ml). After 10 min incubation, 20 ul of nanoparticles at different concentrations were introduced in a final volume of 200 ul. After 24 h, the supernatants were collected and mixed with Quanti-Blue. TLR activation associated with SEAP activity was determined with a multiwell plate reader by measuring the OD at 620 nm. The cell densities (in a 96 well plate) used were 8×10⁴ cells/well for HEK-Blue hTLR9 cells, 5×10⁴ cells/well for HEK-Blue hTLR3 cells, 4×10⁴ cells/well for HEK-Blue hTLR8 cells. Using these methods, the MnO nanoparticles as provided herein have been validated on a wide spectrum of TLRs cells, including HEK-Blue hTLR3, hTLR8 and hTLR9. The nanoparticles are shown to inhibit CpG-induced activation of HEK-Blue hTLR9 cells, inhibit poly (I:C)-induced activation of HEK-Blue hTLR3 cells, and ORN06/Lyo Vec (ORN)-induced activation of HEK-Blue hTLR8 cells in a dose-dependent manner, regardless of the presence or absence of FBS (FIG. 3B-D).

### Plaque Neutralization Reduction Assay

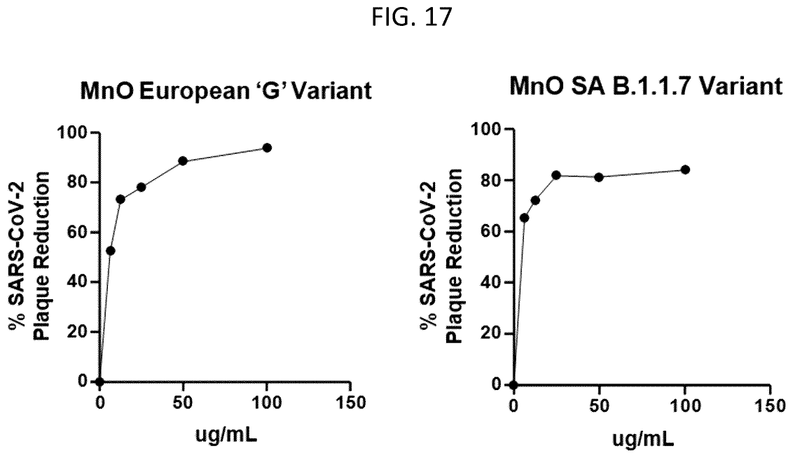

Finally, a plaque reduction neutralization test (PRNT) was used to show that the MnO nanoparticles can bind to SARS-CoV-2 and prevent viral entry, replication, and subsequent cell death in a dose dependent manner. The plaque reduction assay was done using standard methods available to those of skill in the art. Briefly, Vero E6 cells were plated at 1×105 cells/well in a 24 well plate in standard medium overnight and serial dilutions of the MnO nanoparticles were added to the wells starting at 100 ug/mL and two-fold dilutions were made. A negative control was included. Sars-CoV-2 was then added at 50 pfu/well and incubated for 30 minutes. For the assay, 1 mL of 1.2% methylcellulose in 2×MEM was added to each well and the assay was incubated for 4 days. The overlay was removed the plates fixed with 10% NBF and stained with Crystal Violet. The plaques were then counted for each well and all wells were completed in duplicate. Importantly, the MnO nanoparticles were shown to neutralize multiple strains of SARS-CoV-2, including the European “G” variant and the South African “B.1.1.7” variant (FIG. 17). Using this approach, a pan-SARs-CoV-2 inhibitor that is able to limit viral uptake and replication regardless of strain was established. The implications of this discovery provide a potent means of tackling the coronavirus pandemic. A nanoparticle based therapeutic can be delivered intranasally and limit the uptake and replication of any SARs-CoV-2 variant. Our therapy can also be stored stably at 4 degrees C. This can have an immeasurable impact for healthcare workers who need protection in addition to vaccination due to their near constant exposure to all variants of SARs-CoV-2. It also has the potential to provide a means of protection to those that do not have access to vaccines that require storage at −80 C.

### Discussion:

In this study, we performed immune and molecular assays that demonstrated that elevated levels of nucleic acid-containing DAMPs/PAMPs are present in COVID-19 ICU patient serum and ETA. We also observed a monocytic TLR tolerance phenotype in deceased COVID-19 ICU patients. We established that two biomaterials could potentially be used to mitigate DAMP/PAMP-mediated hyperinflammation. TLR activation analyses using patient serum and ETA revealed systemic and local inflammation as indicated by potent stimulation of TLR3, TLR4, TLR7, and TLR9 signaling. DAMPs and PAMPs, which were particularly high in the airways, likely spill into the systemic circulation, resulting in hyperinflammation via persistent TLR stimulation. We observed that, compared to deceased COVID-19 ICU patients, those that recover have a higher abundance of CD16⁺ monocytes, which are known to be responsive to TLR7/8 activation by viral ssRNA. We characterized the functional differences in these monocytes and found that patients with different clinical outcomes mount a differential response to prototypic viral and bacterial PAMPs [31]. To characterize the monocytes in ICU survivors and non-survivors, we measured the response of monocyte subsets following activation with TLR agonists and assayed the serum proteome from patients. These immune profiling, transcriptomic and proteomic analyses all suggested that nucleic acid-containing DAMPs/PAMPs play a central pathological role in the critically ill COVID-19 ICU patients. We therefore explored whether nucleic acid-scavenging agents could be utilized to counteract such DAMPs/PAMPs in samples from these ICU patients. We describe two complementary biomaterial-based approaches, microfibers and soluble nanoparticles, for neutralizing the activation potential of these hyperinflammatory lung and blood samples.

By stimulating isolated monocytes from deceased and recovered patients with TLR agonists, we discovered that cells with the same cytometric phenotype contain distinct molecular biomarkers and undergo differential transcriptional responses that distinguish between patients with different clinical outcomes. Overall, monocytes from recovered patients produce a robust anti-viral response to TLR activation, whereas monocytes from deceased patients amplify the effects of the cytokine storm observed in COVID-19 patients via expression of TNF-α and alarmin mRNAs. These molecular patterns observed in deceased patients are congruent with the concept of TLR tolerance, marked by the lack of TL-12 production induced by the MAPK and AP-1 signaling pathway [[15], [32], [33], [34], [35], [36]]. Indeed, we identified transcriptomic profiles consistent with the previous characterization of IL-12 production in innate myeloid cells from mice in which TLR tolerance was induced with a primary viral infection and a secondary bacterial infection [79,80]. However, CD16⁺ monocytes from deceased ICU patients with COVID-19 had reduced expression of JUND at baseline and did not activate the AP-1 signaling pathway upon stimulation with LPS or R848 (data not shown). This observation suggests that tolerant monocytes lose the ability to produce IL-12 and leave the patients at increased risk of secondary bacterial infection [37,40,81]. Therefore, increased and prolonged inflammation during severe SARS-CoV-2 infection can engender an immunosuppressed phenotype that prevents adequate response to subsequent infections which can lead to poor clinical outcomes. Our data show that deceased patients not only have TLR tolerant monocytes, but that a second independent group of deceased patients has significantly elevated proteinaceous markers of severe bacterial infection. Our proteomics data reveal that patients who eventually died in the ICU had significantly lower levels of PAMP and DAMP carrier proteins in their blood such as MARCO and apolipoproteins, including APOA1, APOA2, APOH, and APOD, especially at later time points of infection. MARCO and apolipoproteins have been shown to protect against bacterial infection due to their ability to bind to PAMPs and transport them to immune cells as well as neutralize LPS to temper cytokine storms [[73], [74], [75], [76], [77]]. A decrease in the circulating levels of these factors most likely results from the consumption of PAMP carriers due to the prolonged abundance of their ligands, which is consistent with TLR hyper-activation and likely predisposes these patients to secondary bacterial infection.

Unfortunately, previous studies have shown that it is difficult to rescue monocytes that have become TLR tolerant [37]. Therefore, a therapeutic strategy designed to treat or stop COVID-19 patients from becoming critically ill will almost assuredly need to prevent the development of TLR tolerance in the first place. With this in mind, we evaluated a strategy to neutralize TLR agonists using nucleic acid-binding microfibers and nanoparticles, effectively supplementing the function of the depleted endogenous PAMP scavengers MARCO and apolipoprotein. As we and others have shown in models of trauma, sepsis, cancer, influenza and now COVID-19, neutralizing or removing DAMPs and PAMPs from circulation using nucleic acid-binding biomaterials can reduce systemic inflammation and TLR tolerance by preventing TLR activation [[11], [12], [13], [14], [15], [16], [17], [21], [22]]. The microfiber mesh strategy can theoretically be modified for use in a variety of devices, including filters for dialysis and extracorporeal membrane oxygenation (ECMO) that are commonly used to treat ICU patients with COVID-19 [82]. MnO-based nanoscavengers can be used for systemic or inhalation delivery in patients before the need for invasive airway support. Patients that are intubated due to virally induced respiratory failure are predisposed to a secondary ventilator-associated bacterial pneumonia (VAP) [[83], [84], [85]]. These bacteria are recognized by their surface expression of the virulence factor LPS. A secondary immune challenge with LPS in mice caused a severe inflammatory reaction and high mortality, defining a similar trajectory to those observed in ICU patients who die from COVID-19 [81,86]. TLR tolerance leads to the inability of monocytes to adequately respond to subsequent immune challenges, as in the case of VAP associated with COVID-19 and poor outcomes. MnO might prove useful to supplement and replenish endogenous scavengers that are being overrun by PAMPs/DAMPs in the critically ill with COVID-19. As shown in FIG. 15, MnO nanoscavengers limit TLR stimulation by COVID-19 patient samples. By limiting TLR stimulation, such nanoparticles should abrogate TLR tolerance of patient monocytes caused by TLR hyperstimulation.

The in vivo translation of this DAMP/PAMP neutralization concept now needs to be tested to determine it can improve outcomes in the setting of COVID-19 infection as has been observed in other animal models of disease [[11], [12], [13], [14], [16], [17]]. Of particular relevance to SARS-CoV-2 infection, we have shown that neutralization of DAMPs/PAMPs improves outcomes in animal models of influenza and sepsis [13,19,20]. Future studies using a large animal model of COVID-19 that recapitulates the TLR tolerance phenotype observed in COVID-19 ICU patients should be pursued to allow for the establishment of an ECMO animal model in which the utility of the proposed microfiber-based DAMP/PAMP capture strategy could be characterized. We did not examine whether similar TLR-tolerant monocytes are associated with patients with non-COVID-19 ARDS but hypothesize that a common immunopathology may underlie dysfunction in sepsis and other severe infections, as well. Furthermore, we were unable to investigate the role of neutrophils and platelets, which also express TLRs and have been implicated in COVID-19 thromboinflammatory syndromes. Future studies using fresh blood are needed to compare the contributions of these cells to the TLR-mediated inflammatory response in COVID-19 and other infectious diseases and the potential utility of microfiber and nanoparticle-based DAMP/PAMP scavengers to control such hyperinflammation.

In summary, we observe that nucleic acid-containing DAMPs/PAMPs are highly elevated in the lungs and blood of COVID-19 ICU patients and that CD16⁺ monocytes isolated from such patients are defined by TLR activation phenotypes that reflect the clinical outcome. Specifically, patients that recover from SARS-CoV-2 infection have CD16⁺ monocytes capable of producing a competent anti-viral immune response through activation of TLRs such as TLR7/8, while cells from deceased patients are tolerized to TLR activation due to chronic, repeated DAMP/PAMP-induced inflammation. We observed that monocytes from deceased patients have transcriptomic and proteinaceous markers characteristic of TLR tolerance consistent with the observation that nucleic acid DAMPs/PAMPs are highly elevated in patient serum and ETA. We tested two complementary approaches to neutralize such inflammatory mediators that, if developed appropriately, might prevent hyperinflammation and monocytic TLR tolerance and improve clinical outcomes for individuals who are severely ill with COVID-19.

### Materials and Methods

### COVID-19 ICU Study Population

The study was approved by the Duke University Institutional Review Board (IRB) (Pro 00101196). Eligible patients were men and women ages 18 years and above that were admitted to an adult ICU with SARS-CoV-2 infection confirmed by PCR testing. Informed consent was obtained from the patient or their legally authorized representative (LAR).

### Duke ICU COVID-19 Biorepository

We collected whole blood and endotracheal aspirate (ETA) supernatants. Whole blood was separated into serum, citrated plasma, EDTA (ethylenediaminetetraacetic acid) plasma, and peripheral blood mononuclear cells (PBMCs). ETA was spun down at 600 g for 10 min at 4° C. The supernatant was pipetted off and the pellet and supernatant were stored at −80° C. Samples were collected on study days 1, 3, 7, 14, and 21. All data reported in this paper were obtained with patient samples from the Duke ICU Biorepository and this study was performed in collaboration with the Biorepository team. The patients involved in this study had an average age of 59.3 years and 61/67 had at least one coexisting condition.

### Healthy Controls

Whole blood was collected from healthy donors under a separate protocol approved by the Duke IRB (Pro 00007265) and processed similarly.

### Reagents

Pooled human serum from healthy donors was purchased from Sigma Aldrich. CpG ODN 2006, poly I:C, LPS, and R848 were purchased from InvivoGen. ELISA kits were purchased as follows: HMGB-1 (Tecan-ST51011), Cell-Death Detection ELISA Plus (Roche-11,774,425,001), and albumin (Abcam-abl79887). Picogreen and Ribogreen stains were obtained from Life Technologies (P7589 and R41190). PAMAM-G3 based nucleic acid-binding fiber and polyethylenimine (PEI)-based nucleic acid-binding fiber were graciously provided by Jaewoo Lee, PhD, and synthesized as previously described [15].

Synthesis of Nucleic Acid-Binding Fiber and Removal of TLR Ligands from Serum and ETA

Electrospinning was utilized to generate nucleic acid-binding microfibers as previously described [15]. Briefly, poly (styrene-alt-maleic anhydride) (PSMA) polymers meshes were used to functionally immobilize [87,88] PAMAM-G3 or PEI onto microfiber meshes containing a mixture of PSMA and polystyrene copolymers. PSMA (0.3 g) (Sigma, Saint Louis, MO) and polystyrene (0.4 g) (Sigma) were dissolved at room temperature in a 1:1:1 (v:v:v) mixture of tetrahydrofuran:acetone:dimethylformamide (3 mL) (Sigma) and microfibers were generated by electrospinning 2 mL of copolymer solution at a dispensing rate of 2 mL/h with an applied voltage of ˜17.3 kV, as described previously [87, 89]. To generate microfiber meshes, the microfibers were collected on a grounded cylindrical mandrel as described [15]. To immobilize PEI and PAMAM-G3 on the PSMA/polystyrene microfiber meshes 1.8 kDa branched polyethylenimine (PEI) (Polysciences, Warrington, PA) (0.005 M) and PAMAM-G3 (0.004 M) (Sigma) were incubated for 72 h either at room temperature (PEI) or 4° C. (PAMAM-G3) as described [15].

Patient sera [10 μL] or ETA [1 μL] were diluted into a final volume of 100 μL with serum-free AIM-V media. Dilutions were incubated with PAMAM-G3-based fiber at 37° C. for 30 min with rotation. The suspension was removed and combined with a PEI-based fiber and a PAMAM-based fiber for 30 min each at 37° C. with rotation prior to use in TLR reporter assays. This sequential fiber-based strategy was validated as the most effective scavenging fiber approach in previous studies [15].

MnO Nanoparticle Synthesis and Neutralization of TLR Ligands from Serum and ETA Using MnO Nanoparticles

Manganese oxide can be synthesized by using manganese compounds (e.g., manganese acetate) and acid (e.g., tannic acid) at high temperature (e.g., 100-150° C.). A mixture of manganese acetate and tannic acid (mass ratio of manganese acetate and tannic acid is 1:2-6) in Milli-Q water is stirred for 10 min at room temperature, the solution is then transferred into an autoclave. After heat treatment at 150° C. for 2 h, the sample solution is cooled to <50° C. naturally. The size of MnO nanoparticles ranged from 30 to 100 nm and zeta potential is about −20 mV.

Patient sera [10 μL] or ETA [1 μL] were diluted into a final volume of 100 μL with serum-free AIM-V media. Dilutions were incubated with MnO at 37° C. for 30 min with rotation prior to use in TLR reporter assays and monocyte stimulation assays.

### Whole Blood Flow Cytometry

Immune subset profiling antibody panels were obtained from Beckman-Coulter. One basic immune subset panel tube (B53309) and one granulocyte panel tube (B88651) was used per patient, per time point per the manufacturer's instructions. The fixed and stained cells were acquired within two days of collection by The Duke Immune Profiling Core (DIPC) in accordance with BSL2* biosafety practices. Data were analyzed using FlowJo.

### TLR Activation Assays

HEK-Blue human TLR 3, 4, 7, and 9 reporter cell lines were purchased from InvivoGen (hkb-htlr3, hkb-htlr4, hkb-htlr7, hkb-tlr9). Activation was determined according to the manufacturer's instructions using QUANTI-blue SEAP detection media (InvivoGen). Cells were plated in 96-well plates at a density of 40,000 cells/well and treated for 12 h with media, low molecular weight (LMW) polyinosinic:polycytidylic acid (polyL:C) [1 μg/mL], lipopolysaccharide (LPS) [1 μg/mL], R848 (Resiquimod) [1 μg/mL], or CpG ODN 2006 [1 μM], pooled healthy human sera [10 μL], COVID-19 patient sera [10 μL], or COVID-19 patient ETA [2 μL] in a final volume of 100 μL media. Cell supernatant was collected and mixed with QUANTI-blue at a 20:80 vol:vol ratio and incubated for 90 min at 37° C. Absorbance was measured using a Spectramax i3 plate reader (Molecular Devices) at 655 nm. All samples were run in triplicate and results are reported as comparative absorbance values.

Plasma Proteomics with COVID-19 ICU Patient Specimens

Samples were thawed, and 20 μL of plasma was aliquoted into 750 μL Matrix tubes (Thermo) in a 96-well format. A study pool QC (SPQC) sample was made by mixing equal volumes of all samples. Three replicates of the SPQC sample were added to the plate. Samples were diluted with 200 of 5.5% w/v sodium deoxycholate (SDC) in 50 mM ammonium bicarbonate (AmBic) containing 11 mM dithiothreitol, and samples were inactivated by heating at 80° C. for 15 min. After cooling, samples were alkylated with 10 μL of 250 mM iodoacetamide (IAM) in AmBic at room temperature in the dark, for 30 min. Alkylation was quenched by addition of 10 μL of 220 mM DTT in AmBic, and digestions were performed by adding 20 μL of 5 mg/mL TPCK trypsin (Sigma) in AmBic followed by incubation at 37° C. for 2 h in a Thermomixer. Reactions were quenched by addition of 30 μL of 5/20/65 v/v/v TFA/MeCN/water containing 0.33 pmol yeast ADH1 digest (Waters MassPrep) to each sample followed by brief vortexing and incubation on a Thermocycler for 5 min and centrifugation for 2 min at 10,000×g. 15,000×g for 2 min, supernatants and residual precipitate were transferred to an ISOLUTE Filtration+filter plate (Biotage) taped to a Deepwell 96/1000 μl plate (Eppendorf) followed by shaking at 1250 rpm for 3 min. Finally, the samples were filtered for 10 min on a vacuum manifold in a 96-well plate and sealed with a cap mat.

Quantitative data-independent acquisition (DIA)-LC-MS/MS: Samples were analyzed by DIA-LCMS/MS using an Acquity UPLC (Waters) interfaced to a Exploris 480 high-resolution tandem mass spectrometer (Thermo). Analyses of plasma samples used 10 μL of peptide digests (˜33 μg). After direct injection, peptides were separated on a 1 mm×15 mm 1.7 m CSH C18 column (Waters) using a flow rate of 100 μL/min, a column temperature of 55° C. and a gradient using 0.1% (v/v) formic acid (FA) in H₂O (mobile phase A) and 0.1% (v/v) FA in MeCN (mobile phase B) as follows: 0-60 min, 3-28% B; 60-60.5 min, 28-90% B; 60.5-62.5 min, 90% B; 62.5-63 min, 90-3% B; and 63-67 min, re-equilibration at 3% B. A tee was used post-column to introduce a solution of 50% (v/v) dimethyl sulfoxide/acetonitrile (DMSO/MeCN) at 10 μL per min. The LC was interfaced to the MS with an Optamax NG ion source under heated electrospray ionization (HESI) conditions with the following tune parameters: sheath gas, 32; aux gas, 5; spray voltage, 3.5 kV; capillary temperature, 275° C.; aux gas heater temp, 125° C.

The DIA analysis used a 120,000 resolution precursor ion (MS1) scan from 375 to 1600 m/z, AGC target of 300% and maximum injection time (IT) of 45 milliseconds and RF lens of 40%; data were collected in centroid mode. MS/MS was performed using tMS2 method with default charge state=3, 30,000 resolution, AGC target of 1000% and maximum IT of 60 ms, and a NCE of 30; data were collected in centroid mode. 18 variable DIA windows spanned 400-1200 m/z. The MS cycle time was 1.65 s, and the total injection-to-injection time was 67 min.

High pH-reversed phase (HPRP) fractionation for spectral library generation: Approximately 500 μg of protein digests, pooled from individual samples, was lyophilized and resuspended in 20 mM ammonium formate, pH 10. Peptides were fractionated using a 2.1 mm×5 cm BEH C18 column (Waters) and Waters ACQUITY I-Class UPLC. Separations utilized a flow rate of 0.4 mL/min and column temperature of 55° C., and mobile phases consisted of 20 mM ammonium formate, pH 10 (MPA) and neat MeCN (MPB). A gradient was as follows: 0-3, 3% MPB; 3-50 min, 3-35% MPB; 50-51 min, 35-90% MPB; 51-55 min, 90% MPB; 55-56 min, 90-3% MPB; 56-70 min, 3% MPB. 48 equal fractions were collected from 3 to 53 min (˜0.4 mL each fraction) into wells containing 10 μl of 40% TFA, and each column of wells (e.g. wells/fractions 1, 13, 25, and 37) was combined to maximize the peak capacity of the LCMS/MS analyses. After lyophilization, samples were resuspended in 25 μl of 1% (v/v) TFA/2% (v/v) MeCN in H₂O.

Qualitative analysis of HPRP-fractionated samples for spectral library generation: 20 μL of each of the fractionated pools (12 fractions each) were analyzed by microflow LC-MS/MS as described above, except that the data was analyzed in Spectronaut 14 using the study-specific library. To minimize missing data, “profiling” was used to quantify precursors that did not meet a significance value (q-value<0.01) in every run. Using a sparse cut-off of 20% (data included all precursors that met a q-value in at least 20% runs), there were 559 proteins quantified in the plasma samples. Of these, 450 proteins (including ADH1, trypsin, and 3 variant protein entries) were quantified by more than 1 unique precursor, a metric for more confident identification.

Normalization: Raw protein group data (sum of all quantified precursors) was normalized by library size, followed by trimmed mean of M (TMM) normalization. Normalization had little effect on expression measurements, suggesting highly stable sample preparation, data acquisition and overall protein content between the study samples measured.

Data analysis: Normalized data was first evaluated by grouping all patient timepoints by outcome. To narrow down the dataset, we performed multiple T tests with Benjamini-Hochberg (BH) correction and proceeded with proteins meeting the threshold of adjusted p<0.05. Using the average protein measurements by outcome we calculated the log 2 fold changes for each protein and grouped upregulated and downregulated proteins separately. These were analyzed in STRING V 11.0 for full STRING network at a required score of highest confidence (0.900) ignoring disconnected nodes, and g:Profiler (version e103_eg50_p15_68c0e33) using default settings [97,102]. We then proceeded with pathway clustering using Enrichmentmap 3.3 application via Cytoscape 3.8, using downloaded pathway and enrichment data from g:Profiler [103,104]. We narrowed on hits consistent with the most enriched pathway clusters, namely defense/immune cell pathways for upregulated proteins, and wound healing/complement and immunity for downregulated genes.

We further evaluated the hits differences between outcome by matched timepoints at 0, 7, or 14-days using T tests to find out when these proteins were most likely to have differences.

ELISA and nucleic acid quantification ELISA, Picogreen, and Ribogreen assays (Life Sciences) were performed per the manufacturer's instructions in triplicate.

Statistical analysis Statistical tests were performed in GraphPad Prism via one-way ANOVA followed by Sidak multiple comparison test between healthy sera controls and COVID-19 samples or between COVID-19 sample types. All other graphs were created with GraphPad Prism software.

### REFERENCES

1. Medzhitov R. Origin and physiological roles of inflammation. Nature. 2008; 454(7203):428-435.
2. Barton G. M. A calculated response: control of inflammation by the innate immune system. J. Clin. Invest. 2008; 118(2):413.
3. Hansen J. D., Vojtech L. N., Laing K. J. Developmental & Comparative; 2011. Sensing Disease and Danger: a Survey of Vertebrate PRRs and Their Origins.
4. Rakoff-Nahoum S., Medzhitov R. Toll-like receptors and cancer. Nat. Rev. Cancer. 2009; 9(1):57-63.
5. Bhatelia K., Singh K., Singh R. TLRs: linking inflammation and breast cancer. Cell. Signal. 2014; 26(11):2350-2357.
6. Pandey S., Singh S., Anang V., Bhatt A. N., Natarajan K., Dwarakanath B. S. Pattern recognition receptors in cancer progression and metastasis. Cancer Growth Metastasis. 2015; 8:25-34.
7. Mehmeti M., Allaoui R., Bergenfelz C., Saal L. H., Ethier S. P., Johansson M. E., Jirstrom K., Leandersson K. Expression of functional toll like receptor 4 in estrogen receptor/progesterone receptor-negative breast cancer. Breast Cancer Res. 2015; 17(1):130.
8. Gonzalez-Reyes S., Marin L., Gonzalez L., Gonzalez L. O., del Casar J. M., Lamelas M. L., Gonzalez-Quintana J. M., Vizoso F. J. Study of TLR3, TLR4 and TLR9 in breast carcinomas and their association with metastasis. BMC Cancer. 2010; 10:665.
9. Sun S., Sursal T., Adibnia Y., Zhao C., Zheng Y., Li H., Otterbein L. E., Hauser C. J., Itagaki K. Mitochondrial DAMPs increase endothelial permeability through neutrophil dependent and independent pathways. PLoS One. 2013; 8(3).
10. Feng Z., Qi S., Zhang Y., Qi Z., Yan L., Zhou J., He F., Li Q., Yang Y., Chen Q., Xiao S., Li Q., Chen Y., Zhang Y. Ly6G+ neutrophil-derived miR-223 inhibits the NLRP3 inflammasome in mitochondrial DAMP-induced acute lung injury. Cell Death Dis. 2017; 8(11) e3170-e3170.
11. Holl E. K., Shumansky K. L., Borst L. B., Burnette A. D., Sample C. J., Ramsburg E. A., Sullenger B. A. Scavenging nucleic acid debris to combat autoimmunity and infectious disease. Proc. Natl. Acad. Sci. U.S.A 2016; 113(35):9728-9733.
12. Holl E. K., Bond J. E., Selim M. A., Ehanire T., Sullenger B., Levinson H. The nucleic acid scavenger polyamidoamine third-generation dendrimer inhibits fibroblast activation and granulation tissue contraction. Plast. Reconstr. Surg. 2014; 134(3):420-433.
13. Holl E. K., Shumansky K. L., Pitoc G., Ramsburg E., Sullenger B. A. Nucleic acid scavenging polymers inhibit extracellular DNA-mediated innate immune activation without inhibiting anti-viral responses. PLoS One. 2013; 8(7).
14. Naqvi I., Gunaratne R., McDade J. E., Moreno A., Rempel R., Rouse D. C., Herrera S. G., Pisetsky D. S., Lee J., White R. R., Sullenger B. A. Polymer-mediated inhibition of pro-invasive nucleic acid DAMPs and microvesicles limits pancreatic cancer metastasis. Mol. Ther. 2018; 26(4):1020-1031.
15. Eppensteiner J., Kwun J., Scheuermann U., Barbas A., Limkakeng A. T., Kuchibhatla M., Elster E. A., Kirk A. D., Lee J. Damage- and pathogen-associated molecular patterns play differential roles in late mortality after critical illness. Jci Insight. 2019; 4(16).
16. Jain S., Pitoc G. A., Holl E. K., Zhang Y., Borst L., Leong K. W., Lee J., Sullenger B. A. Nucleic acid scavengers inhibit thrombosis without increasing bleeding. Proc. Natl. Acad. Sci. Unit. States Am. 2012; 109(32).
17. Lee J., Sohn J. W., Zhang Y., Leong K. W., Pisetsky D., Sullenger B. A. Nucleic acid-binding polymers as anti-inflammatory agents. Proc. Nat. Acad. Sci. 2011; 108(34):14055-14060. PMID: 21844380.
18. Liang H., Peng B., Dong C., Liu L., Mao J., Wei S., Wang X., Xu H., Shen J., Mao H.-Q., Gao X., Leong K. W., Chen Y. Cationic nanoparticle as an inhibitor of cell-free DNA-induced inflammation. Nat. Commun. 2018; 9(1):4291.
19. Dawulieti J., Sun M., Zhao Y., Shao D., Yan H., Lao Y.-H., Hu H., Cui L., Lv X., Liu F., Chi C.-W., Zhang Y., Li M., Zhang M., Tian H., Chen X., Leong K. W., Chen L. Treatment of severe sepsis with nanoparticulate cell-free DNA scavengers. Sci. Adv. 2020; 6(22).
20. Kelly L., Olson L. B., Rempel R. E., Everitt J. I., Levine D., Nair S. K., Davis M. E., Sullenger B. A. β-Cyclodextrin-containing polymer treatment of cutaneous lupus and influenza improves outcomes. Mol. Ther. 2021; 30:845-854.
21. Holl E. K., Frazier V., Landa K., Boczkowski D., Sullenger B., Nair S. K. Controlling cancer-induced inflammation with a nucleic acid scavenger prevents lung metastasis in murine models of breast cancer. Mol. Ther. 2021; 29(5):1772-1781.
22. Eteshola E. O. U., Landa K., Rempel R. E., Naqvi I. A., Hwang E. S., Nair S. K., Sullenger B. A. Breast cancer-derived DAMPs enhance cell invasion and metastasis, while nucleic acid scavengers mitigate these effects. Mol. Ther. Nucleic Acids. 2021; 26:1-10.
23. Zhou F., Yu T., Du R., Fan G., Liu Y., Liu Z., Xiang J., Wang Y., Song B., Gu X., Guan L., Wei Y., Li H., Wu X., Xu J., Tu S., Zhang Y., Chen H., Cao B. Clinical course and risk factors for mortality of adult inpatients with COVID-19 in Wuhan, China: a retrospective cohort study. Lancet. 2020; 395:1054-1062.
24. Rodriguez-Morales A. J., Cardona-Ospina J. A., Gutierrez-Ocampo E., Villamizar-Pena R., Holguin-Rivera Y., Escalera-Antezana J. P., Alvarado-Arnez L. E., Bonilla-Aldana D. K., Franco-Paredes C., Henao-Martinez A. F., Paniz-Mondolfi A., Lagos-Grisales G. J., Ramirez-Vallejo E., Suarez J. A., Zambrano L. I., Villamil-Gómez W. E., Balbin-Ramon G. J., Rabaan A. A., Harapan H., Dhama K., Nishiura H., Kataoka H., Ahmad T., Sah R., L. A. N.o.C. D. Covid-Research. Travel Med Infect Di; 2020. Clinical, Laboratory and Imaging Features of COVID-19: A Systematic Review and Meta-Analysis; p. 101623.
25. Goh K. J., Choong M. C., Cheong E. H., Kalimuddin S., Wen S. D., Phua G. C., Chan K. S., Mohideen S. H. Rapid progression to acute respiratory distress syndrome: review of current understanding of critical illness from COVID-19 infection. Ann. Acad. Med. Singapore. 2020; 49(1):1-9.
26. Lin L., Lu L., Cao W., Li T. Hypothesis for potential pathogenesis of SARS-CoV-2 infection—a review of immune changes in patients with viral pneumonia. Emerg. Microb. Infect. 2020:1-14.
27. Shigeto H., Tomoya H., Yukihiro A., Naoya M., Taro I., Masafumi S., Hideo H., Osamu T., Kazunori O., Takeshi S., Kazunori T. Identification of neutrophil extracellular traps in the blood of patients with systemic inflammatory response syndrome. J. Int. Med. Res. 2013; 41(1):162-168.
28. Zhang D., Guo R., Lei L., Liu H., Wang Y., Wang Y., Dai T., Zhang T., Lai Y., Wang J., Liu Z., He A., O'Dwyer M., Hu J. Medrxiv; 2020. COVID-19 Infection Induces Readily Detectable Morphological and Inflammation-Related Phenotypic Changes in Peripheral Blood Monocytes, the Severity of Which Correlate with Patient Outcome; p. 2020. 03.24.20042655.
29. Zhou Y., Fu B., Zheng X., Wang D., Zhao C., Qi Y., Sun R., Tian Z., Xu X., Wei H. National Science Review; 2020. Pathogenic T-Cells and Inflammatory Monocytes Incite Inflammatory Storms in Severe COVID-19 Patients.
30. Ka M. B., Olive D., Mege J.-L. Modulation of monocyte subsets in infectious diseases. World J. Immunol. 2014; 4(3):185.
31. Cros J., Cagnard N., Woollard K., Patey N., Zhang S.-Y., Senechal B., Puel A., Biswas S. K., Moshous D., Picard C., Jais J.-P., D'Cruz D., Casanova J.-L., Trouillet C., Geissmann F. Human CD14dim monocytes patrol and sense nucleic acids and viruses via TLR7 and TLR8 receptors. Immunity. 2010; 33(3):375-386.
32. Tsukada K., Kitazawa T., Fukushima A., Okugawa S., Yanagimoto S., Tatsuno K., Koike K., Nagase H., Hirai K., Ota Y. Macrophage tolerance induced by stimulation with Toll-like receptor 7/8 ligands. Immunol. Lett. 2007; 111(1):51-56.
33. Ziegler-Heitbrock H. W., Wedel A., Schraut W., Strobel M., Wendelgass P., Sternsdorf T., Bauerle P. A., Haas J. G., Riethmuller G. Tolerance to lipopolysaccharide involves mobilization of nuclear factor kappa B with predominance of p50 homodimers. J. Biol. Chem. 1994; 269(25):17001-17004.
34. Broad A., Jones D., Kirby J. Toll-like receptor (TLR) response tolerance: a key physiological “damage limitation” effect and an important potential opportunity for therapy. Curr. Med. Chem. 2006; 13(21):2487-2502.
35. Butcher S. K., O'Carroll C. E., Wells C. A., Carmody R. J. Toll-like receptors drive specific patterns of tolerance and training on restimulation of macrophages. Front. Immunol. 2018; 9:933.
36. Broad A., Kirby J. A., Jones D. E. J. Toll-like receptor interactions: tolerance of MyD88-dependent cytokines but enhancement of MyD88-independent interferon-β production. Immunology. 2007; 120(1):103-111.
37. Weighardt H., Heidecke C.-D., Emmanuilidis K., Maier S., Bartels H., Siewert J.-R., Holzmann B. Sepsis after major visceral surgery is associated with sustained and interferon-γ-resistant defects of monocyte cytokine production. Surgery. 2000; 127(3):309-315.
38. Altare F., Lammas D., Revy P., Jouanguy E., Döffinger R., Lamhamedi S., Drysdale P., Scheel-Toellner D., Girdlestone J., Darbyshire P., Wadhwa M., Dockrell H., Salmon M., Fischer A., Durandy A., Casanova J.-L., Kumararatne D. S. Inherited interleukin 12 deficiency in a child with bacille Calmette-Guérin and Salmonella enteritidis disseminated infection. J. Clin. Invest. 1998; 102(12):2035-2040.
39. Haraguchi S., Day N. K., Nelson R. P., Emmanuel P., Duplantier J. E., Christodoulou C. S., Good R. A. Interleukin 12 deficiency associated with recurrent infections. Proc. Natl. Acad. Sci. Unit. States Am. 1998; 95(22):13125-13129.
40. Hensler T., Heidecke C. D., Hecker H., Heeg K., Bartels H., Zantl N., Wagner H., Siewert J. R., Holzmann B. Increased susceptibility to postoperative sepsis in patients with impaired monocyte IL-12 production. J. Immunol. 1998; 161(5):2655-2659.
41. Bhatraju P. K., Ghassemieh B. J., Nichols M., Kim R., Jerome K. R., Nalla A. K., Greninger A. L., Pipavath S., Wurfel M. M., Evans L., Kritek P. M., West T. E., Luks A., Gerbino A., Dale C. R., Goldman J. D., O'Mahony S., Mikacenic C. Covid-19 in critically ill patients in the seattle region—case series. N. Engl. J. Med. 2020; 382:2012-2022.
42. Liu Y., Du X., Chen J., Jin Y., Peng L., Wang H. H. X., Luo M., Chen L., Zhao Y. Neutrophil-to-lymphocyte ratio as an independent risk factor for mortality in hospitalized patients with COVID-19. J. Infect. 2020; 81:6-12.
43. Liu Z., Long W., Tu M., Chen S., Huang Y., Wang S., Zhou W., Chen D., Zhou L., Wang M., Wu M., Huang Q., Xu H., Zeng W., Guo L. Lymphocyte subset (CD4+, CD8+) counts reflect the severity of infection and predict the clinical outcomes in patients with COVID-19. J. Infect. 2020; 81:318-356.
44. Phua J., Weng L., Ling L., Egi M., Lim C.-M., Divatia J. V., Shrestha B. R., Arabi Y. M., Ng J., Gomersall C. D., Nishimura M., Koh Y., Du B., Group A. C. C. C. T. Intensive care management of coronavirus disease 2019 (COVID-19): challenges and recommendations. Lancet Respir. Med. 2020; 8:506-517.
45. Poston J. T., Patel B. K., Davis A. M. Management of critically ill adults with COVID-19. JAMA. 2020; 323(16).
46. Qin C., Zhou L., Hu Z., Zhang S., Yang S., Tao Y., Xie C., Ma K., Shang K., Wang W., Tian D.-S. Clin Infect Dis Official Publ Infect Dis Soc Am; 2020. Dysregulation of Immune Response in Patients with COVID-19 in Wuhan, China.
47. Ruan Q., Yang K., Wang W., Jiang L., Song J. Clinical predictors of mortality due to COVID-19 based on an analysis of data of 150 patients from Wuhan, China. Intensive Care Med. 2020:1-3.
48. Srikrishna G., Freeze H. H. Endogenous damage-associated molecular pattern molecules at the crossroads of inflammation and cancer. Neoplasia. 2009; 11(7):615-628.
49. Ivanov S., Dragoi A.-M. M., Wang X., Dallacosta C., Louten J., Musco G., Sitia G., Yap G. S., Wan Y., Biron C. A., Bianchi M. E., Wang H., Chu W.-M. M. A novel role for HMGB1 in TLR9-mediated inflammatory responses to CpG-DNA. Blood. 2007; 110(6):1970-1981.
50. Haghbin M., Rostami-Nejad M., Forouzesh F., Sadeghi A., Rostami K., Aghamohammadi E., Asadzadeh-Aghdaei H., Masotti A., Zali M. R. The role of CXCR3 and its ligands CXCL10 and CXCL11 in the pathogenesis of celiac disease. Medicine. 2019; 98(25) e15949-e15949.
51. Shaath H., Vishnubalaji R., Elkord E., Alajez N. M. Single-cell transcriptome analysis highlights a role for neutrophils and inflammatory macrophages in the pathogenesis of severe COVID-19. Cells. 2020; 9(11) 2374-2374.
52. Gauss K. A., Nelson-Overton L. K., Siemsen D. W., Gao Y., DeLeo F. R., Quinn M. T. Role of NF-κB in transcriptional regulation of the phagocyte NADPH oxidase by tumor necrosis factor-α J. Leukoc. Biol. 2007; 82(3):729-741.
53. Khanmohammadi S., Rezaei N. Role of Toll-like receptors in the pathogenesis of COVID-19. J. Med. Virol. 2021; 93(5):2735-2739.
54. Mahler M., Meroni P.-L., Infantino M., Buhler K. A., Fritzler M. J. Circulating calprotectin as a biomarker of COVID-19 severity. Expet Rev. Clin. Immunol. 2021; 17(5):431-443.
55. Forster R., Davalos-Misslitz A. C., Rot A. CCR7 and its ligands: balancing immunity and tolerance. Nat. Rev. Immunol. 2008; 8(5):362-371.
56. Higuchi H., Shoji T., Iijima S., Nishijima K.-i. Constitutively expressed Siglec-9 inhibits LPS-induced CCR7, but enhances IL-4-induced CD200R expression in human macrophages. Biosci. Biotechnol. Biochem. 2016; 80(6):1141-1148.
57. Li S., Miao T., Sebastian M., Bhullar P., Ghaffari E., Liu M., Symonds A. L. J., Wang P. The transcription factors Egr 2 and Egr3 are essential for the control of inflammation and antigen-induced proliferation of B and T cells. Immunity. 2012; 37(4):685-696.
58. Chevrier N., Mertins P., Artyomov M. N., Shalek A. K., Iannacone M., Ciaccio M. F., Gat-Viks I., Tonti E., DeGrace M. M., Clauser K. R., Garber M., Eisenhaure T. M., Yosef N., Robinson J., Sutton A., Andersen M. S., Root D. E., von Andrian U., Jones R. B., Park H., Carr S. A., Regev A., Amit I., Hacohen N. Systematic discovery of TLR signaling components delineates viral-sensing circuits. Cell. 2011; 147(4):853-867.
59. Saichi M., Ladjemi M. Z., Korniotis S., Rousseau C., Ait Hamou Z., Massenet-Regad L., Amblard E., Noel F., Marie Y., Bouteiller D., Medvedovic J., Pene F., Soumelis V. Single-cell RNA sequencing of blood antigen-presenting cells in severe COVID-19 reveals multi-process defects in antiviral immunity. Nat. Cell Biol. 2021; 23(5):538-551.
60. Zhang C., Bai N., Chang A., Zhang Z., Yin J., Shen W., Tian Y., Xiang R., Liu C. ATF4 is directly recruited by TLR4 signaling and positively regulates TLR4-trigged cytokine production in human monocytes. Cell. Mol. Immunol. 2013; 10(1):84-94.
61. Cohen D. M., Won K.-J., Nguyen N., Lazar M. A., Chen C. S., Steger D. J. ATF4 licenses C/EBPP activity in human mesenchymal stem cells primed for adipogenesis. Elife. 2015; 4.
62. Kawai T., Akira S. TLR signaling. Semin. Immunol. 2007; 19(1):24-32.
63. Kawai T., Akira S. Signaling to NF-kappaB by toll-like receptors. Trends Mol. Med. 2007; 13(11):460-469.
64. Kaisho T., Akira S. Toll-like receptor function and signaling. J. Allergy Clin. Immunol. 2006; 117(5):979.
65. Kawai T., Akira S. Signaling to NF-kappaB by toll-like receptors. Trends Mol. Med. 2007; 13(11):460-469.
66. Adelaja A., Hoffmann A. Signaling crosstalk mechanisms that may fine-tune pathogen-responsive NFκB. Front. Immunol. 2019; 10.
67. Borregaard N., Sørensen O. E., Theilgaard-.M. K. Neutrophil granules: a library of innate immunity proteins. Trends Immunol. 2007; 28(8):340-345.
68. Prince L. R., Whyte M. K., Sabroe I., Parker L. C. The role of TLRs in neutrophil activation. Curr. Opin. Pharmacol. 2011; 11(4):397-403.
69. Hayashi F., Means T. K., Luster A. D. Blood; 2003. Toll-like Receptors Stimulate Human Neutrophil Function.
70. Krisztina F., Szabina F., Attila M. Neutrophil cell surface receptors and their intracellular signal transduction pathways. Int. Immunopharm. 2013; 17(3).
71. Flo T. H., Smith K. D., Sato S., Rodriguez D. J., Holmes M. A., Strong R. K., Akira S., Aderem A. Lipocalin 2 mediates an innate immune response to bacterial infection by sequestrating iron. Nature. 2004; 432(7019):917-921.
72. Geetha S. S100A8 and S100A9: new insights into their roles in malignancy. J. Innate Immun. 2011; 4(1):31-40.
73. Mukhopadhyay S., Varin A., Chen Y., Liu B., Tryggvason K., Gordon S. SR-A/MARCO-mediated ligand delivery enhances intracellular TLR and NLR function, but ligand scavenging from cell surface limits TLR4 response to pathogens. Blood. 2011; 117(4):1319-1328.
74. Komai K., Shichita T., Ito M., Kanamori M., Chikuma S., Yoshimura A. Role of scavenger receptors as damage-associated molecular pattern receptors in Toll-like receptor activation. Int. Immunol. 2017; 29(2):59-70.
75. Jiao Y.-., Wu M.-P. Apolipoprotein A-I diminishes acute lung injury and sepsis in mice induced by lipoteichoic acid. Cytokine. 2008; 43(1):83-87.
76. Georgila K., Vyrla D., Drakos E. Apolipoprotein A-I (ApoA-I), immunity, inflammation and cancer. Cancers. 2019; 11(8):1097.
77. Wurfel M. M., Kunitake S. T., Lichenstein H., Kane J. P., Wright S. D. Lipopolysaccharide (LPS)-binding protein is carried on lipoproteins and acts as a cofactor in the neutralization of LPS. J. Exp. Med. 1994; 180(3):1025-1035.
78. Monguió-Tortajada M., Franquesa M., Sarrias M.-R., Borris F. E. Low doses of LPS exacerbate the inflammatory response and trigger death on TLR3-primed human monocytes. Cell Death Dis. 2018; 9(5):499.
79. Dong C., Davis R. J., Flavell R. A. Map kinases in the immune response. Annu. Rev. Immunol. 2002; 20(1):55-72.
80. Lu H. T., Yang D. D., Wysk M., Gatti E., Mellman I., Davis R. J., Flavell R. A. Defective IL-12 production in mitogen-activated protein (MAP) kinase kinase 3 (Mkk3)-deficient mice. EMBO J. 1999; 18(7):1845-1857.
81. Nansen A., Thomsen A. R. Viral infection causes rapid sensitization to lipopolysaccharide: central role of IFN-αβ J. Immunol. 2001; 166(2):982-988.
82. Xu Z., Tang Y., Huang Q., Fu S., Li X., Lin B., Xu A., Chen J. Systematic review and subgroup analysis of the incidence of acute kidney injury (AKI) in patients with COVID-19. BMC Nephrol. 2021; 22(1):52.
83. Luyt C.-E., Sahnoun T., Gautier M., Vidal P., Burrel S., Chambrun M. P. d., Chommeloux J., Desnos C., Arzoine J., Nieszkowska A., Bréchot N., Schmidt M., Hekimian G., Boutolleau D., Robert J., Combes A., Chastre J. Ventilator-associated pneumonia in patients with SARS-CoV-2-associated acute respiratory distress syndrome requiring ECMO: a retrospective cohort study. Ann. Intensive Care. 2020; 10(1):158.
84. Davis K. A. Ventilator-associated pneumonia: a review. J. Intensive Care Med. 2006; 21(4):211-226.
85. Sluijs K. F. v. d., Poll T. v. d., Lutter R., Juffermans N. P., Schultz M. J. Bench-to-bedside review: bacterial pneumonia with influenza—pathogenesis and clinical implications. Crit. Care. 2010; 14(2):219.
86. Nansen A., Christensen J. P., Marker O., Thomsen A. R. Sensitization to lipopolysaccharide in mice with asymptomatic viral infection: role of T cell-dependent production of interferon-γ J Infect. Dis. 1997; 176(1):151-157.
87. Tang C., Ye S., Liu H. Electrospinning of poly(styrene-co-maleic anhydride) (SMA) and water-swelling behavior of crosslinked/hydrolyzed SMA hydrogel nanofibers. Polymer. 2007; 48(15):4482-4491.
88. Stoilova O., Ignatova M., Manolova N., Godjevargova T., Mita D. G., Rashkov I.

Functionalized electrospun mats from styrene-maleic anhydride copolymers for immobilization of acetylcholinesterase. Eur. Polym. J 2010; 46(10):1966-1974.
89. Ignatova M., Stoilova O., Manolova N., Markova N., Rashkov I. Electrospun mats from styrene/maleic anhydride copolymers: modification with amines and assessment of antimicrobial activity. Macromol. Biosci. 2010; 10(8):944-954.
90. Stuart T., Butler A., Hoffman P., Hafemeister C., Papalexi E., Mauck W. M., Hao Y., Stoeckius M., Smibert P., Satija R. Comprehensive integration of single-cell data. Cell. 2019; 177(7):1888-1902. e21. 91. Korsunsky I., Millard N., Fan J., Slowikowski K., Zhang F., Wei K., Baglaenko Y., Brenner M., Loh P.-r., Raychaudhuri S. Fast, sensitive and accurate integration of single-cell data with Harmony. Nat. Methods. 2019; 16(12):1289-1296.
92. Aran D., Looney A. P., Liu L., Wu E., Fong V., Hsu A., Chak S., Naikawadi R. P., Wolters P. J., Abate A. R., Butte A. J., Bhattacharya M. Reference-based analysis of lung single-cell sequencing reveals a transitional profibrotic macrophage. Nat. Immunol. 2019; 20(2):163-172.
93. Alquicira-Hernandez J., Powell J. E. Nebulosa recovers single-cell gene expression signals by kernel density estimation. Bioinformatics. 2021; 37:2485-2487.
94. Robinson M. D., McCarthy D. J., Smyth G. K. edgeR: a Bioconductor package for differential expression analysis of digital gene expression data. Bioinformatics. 2010; 26(1):139-140.
95. McCarthy D. J., Chen Y., Smyth G. K. Differential expression analysis of multifactor RNA-Seq experiments with respect to biological variation. Nucleic Acids Res. 2012; 40(10):4288-4297.
96. Conway J. R., Lex A., Gehlenborg N. UpSetR: an R package for the visualization of intersecting sets and their properties. Bioinformatics. 2017; 33(18):2938-2940.
97. Raudvere U., Kolberg L., Kuzmin I., Arak T., Adler P., Peterson H., Vilo J. g:Profiler: a web server for functional enrichment analysis and conversions of gene lists (2019 update) Nucleic Acids Res. 2019; 47(W1):W191-W198.
98. Fertig E. J., Ding J., Favorov A. V., Parmigiani G., Ochs M. F. CoGAPS: an R/C++ package to identify patterns and biological process activity in transcriptomic data. Bioinformatics. 2010; 26(21):2792-2793.
99. Aibar S., González-Blas C. B., Moerman T., Huynh-Thu V. A., Imrichova H., Hulselmans G., Rambow F., Marine J.-C., Geurts P., Aerts J., van den Oord J., Atak Z. K., Wouters J., Aerts S. SCENIC: single-cell regulatory network inference and clustering. Nat. Methods. 2017; 14(11):1083-1086.
100. Van de Sande B., Flerin C., Davie K., De Waegeneer M., Hulselmans G., Aibar S., Seurinck R., Saelens W., Cannoodt R., Rouchon Q., Verbeiren T., De Maeyer D., Reumers J., Saeys Y., Aerts S. A scalable SCENIC workflow for single-cell gene regulatory network analysis. Nat. Protoc. 2020; 15(7):2247-2276.
101. Kanehisa M. KEGG: kyoto encyclopedia of genes and genomes. Nucleic Acids Res. 2000; 28(1):27-30.
102. Szklarczyk D., Gable A. L., Lyon D., Junge A., Wyder S., Huerta-Cepas J., Simonovic M., Doncheva N. T., Morris J. H., Bork P., Jensen L. J., Mering C.v. STRING v11: protein-protein association networks with increased coverage, supporting functional discovery in genome-wide experimental datasets. Nucleic Acids Res. 2019; 47(D1):D607-D613.
103. Merico D., Isserlin R., Stueker O., Emili A., Bader G. D. Enrichment map: a network-based method for gene-set enrichment visualization and interpretation. PLoS One. 2010; 5(11) e13984-e13984.
104. Shannon P. Cytoscape: a software environment for integrated models of biomolecular interaction networks. Genome Res. 2003; 13(11):2498-2504.

1. A method of reducing or inhibiting viral infection of cells, the method comprising contacting the cells with an anionic manganese oxide (MnO) nanoparticle.

2. The method of claim 1, wherein the cells are in a subject.

3. A method of reducing or inhibiting a viral infection of a subject, the method comprising administering an anionic MnO nanoparticle to the subject.

4. The method of claim 3, wherein the subject is at high risk of exposure to a virus.

5. The method of claim 3, wherein the subject is believed to have been exposed to a virus.

6. The method of claim 3, wherein the subject has been infected by a virus.

7. The method of claim 3, wherein the viral infection is caused by SARS-CoV-2.

8. A method of reducing viral infection induced inflammation in a subject, the method comprising administering an anionic MnO nanoparticle to the subject.

9. The method of claim 8, wherein the viral infection is caused by SARS-CoV-2.

10. The method of claim 8, wherein the inflammation is due to activation of Toll-like receptor (TLR)-signaling by nucleic acid-containing DAMPs/PAMPs.

11. The method of claim 10, wherein the method prevents activation of one or more nucleic acid-sensing TLRs in the subject.

12. The method of claim 11, wherein the one or more nucleic acid-sensing TLRs comprise TLR3, TLR7, TLR9, and/or TLR4.

13. The method of claim 11, wherein the method prevents the development of TLR tolerant monocytes in the subject after viral infection.

14. The method of claim 8, wherein the method prevents the development of a cytokine storm in response to viral infection.

15. The method of claim 3, wherein the MnO nanoparticle is administered orally, nasally, or topically.

16. The method of claim 3, wherein the MnO nanoparticle is administered intravenously.

17. The method of claim 3, wherein the average particle size of the MnO nanoparticles is 30-100 nm.

18. The method of claim 3, wherein the MnO nanoparticle is made by a method comprising the steps of:
mixing a manganese compound and an acid in water to form a mixture;
stirring the mixture;
heat treating the mixture;
cooling the mixture; and
extracting anionic MnO nanoparticles from the cooled mixture.
